(19) United States
(12) Patent Application Publication
(10) Pub. No.: US 2023/0127559 A1
(43) Pub. Date: Apr. 27, 2023

(54) PEPTIDE ANALOGS AND USE OF SAME IN TREATING DISEASES, DISORDERS OR CONDITIONS ASSOCIATED WITH MUTANT P53 PROTEIN

(71) Applicant: YEDA RESEARCH AND DEVELOPMENT CO. LTD., Rehovot (IL)

(72) Inventors: Varda ROTTER, Rehovot (IL); Moshe OREN, Rehovot (IL); Perry TAL, Rehovot (IL); Fernando ALBERICIO, Rehovot (IL)

(21) Appl. No.: 17/911,277

(22) PCT Filed: Mar. 15, 2021
(86) PCT No.: PCT/IL2021/050280

§ 371 Date: Sep. 13, 2022

Related U.S. Application Data
(60) Provisional application No. 62/990,017, filed on Mar. 16, 2020.

(51) Int. Cl. A61K 38/10; A61K 9/00; A61P 35/00

(52) U.S. Cl. CPC A61K 38/10; A61K 9/0019; A61P 35/00

(57) ABSTRACT

Peptide analogs which are endowed with the property of at least partially reactivating a mutant p53 protein are provided. Also provided are uses thereof in the treatment of diseases associated with mutant p53.

### FIELD OF THE INVENTION

The present invention, in some embodiments thereof, relates to peptide analogs and use of same in treating diseases, disorders or conditions associated with a mutant p53 protein, in particular cancer.

### BACKGROUND OF THE INVENTION

Cancer is a leading cause of death in developed countries, and as the average age of the population continues to rise, so do the numbers of diagnosed cases and economic implications. Cancer is not a single disease, but rather a group of more than 200 diseases characterized by uncontrolled growth and spread of abnormal cells. Cancer is a highly heterogeneous disease with major molecular differences in the expression and distribution of tumor cell surface markers even among patients with the same type and grade of cancer. Moreover, cellular mutations tend to accumulate as cancer progresses, further increasing tumor heterogeneity. Most tumor cells exhibit genomic instability with an increased expression of oncogenes and inactivation of tumor suppressor genes.

The p53 gene is considered to be the most important tumor suppressor gene, which acts as a major barrier against cancer progression. The p53 protein responds to various types of cellular stress, and triggers cell cycle arrest, apoptosis, or senescence. This is achieved by transcriptional transactivation of specific target genes carrying p53 DNA binding motifs. It is widely agreed that the p53 pathway is impaired in almost all human cancers. Mutation of p53 is viewed as a critical step in malignant transformation process and over 50% of cancer cases carry mutations in their p53 genes. Most of these mutations are missense point mutations that target the DNA-binding core domain (DBD) of p53, thereby abolishing specific DNA binding of p53 to its target site. These mutations prevent p53-dependent transcription and consequently p53-mediated tumor suppression.

Structural studies have revealed that the tumor-derived missense mutations in the DBD of p53 produce a common effect: destabilization of DBD folding at physiological temperature (Joerger et al. J Biol Chem, 2004. 279(2): p. 1291-6). Mutant p53 proteins accumulate at high levels in tumor cells, mainly due to their inability to upregulate the expression of p53's own destructor Mdm2. Moreover, many p53 activating stress signals (like hypoxia, genomic instability and oncogene expression) are constitutively induced in cancer cells. Therefore, reactivation of Mut-p53 is expected to exert major anti-tumor effects. Furthermore, it has been shown in a mouse model that restoration of p53 functions is well tolerated in normal tissues and produces no visible toxic effects (Ventura, A., et al. Nature, 2007. 445(7128): p. 661-5).

Structural studies show that the extent of misfolding differs among mutants; however, there is no defined alternative fold but rather a partial denaturation. This suggests that a “small molecule” approach to reverse the effect of p53 mutation on folding could be applicable to a wide range of mutant forms. Another important prediction from structural studies is that a ligand that binds to the properly folded fraction of the protein is expected to shift the equilibrium towards the native fold according to the law of mass action.

Several correctional approaches were attempted in the p53 conformation field. Proof of principle for conformation stabilizing peptides was provided by Friedler and colleagues (Friedler, A., et al. Proc. Natl. Acad. Sci. USA, 2002. 99(2): p. 937-42). A nine-residue peptide, CDB3, was designed based on the crystal structure of the complex between the p53 DBD and ASPP (Samuels-Lev, Y., et al., Mol. Cell, 2001. 8(4): p. 781-94). This peptide was shown to bind Mut-p53 and act as a chaperone, shifting equilibrium towards the wildtype (WT) conformation, as indicated by increased reactivity to the monoclonal antibody PAb1620. However, the biological effects of CDB3 (Issaeva, N., et al., Proc. Natl. Acad. Sci. USA, 2003. 100(23): p. 13303-7) are only partial since the conformation of the Mut-p53/CDB3 complex is in an intermediate state between WT and mutant.

Small molecule compounds targeting Mut-p53 have been identified using either protein-based or cell-based assays (Peng, Y., et al., Oncogene, 2003. 22(29): p. 4478-87). CP-31398 was identified by screening for molecules that protect the isolated p53 DBD from thermal denaturation, as assessed by maintenance of PAb1620 reactivity upon protein heating (Foster, B. A., et al., Science, 1999. 286(5449): p. 2507-10). The mechanism of action of CP-31398 remains unclear. NMR studies failed to detect any binding of CP-31398 to the p53 DBD (Rippin, T. M., et al., Oncogene, 2002. 21(14): p. 2119-29). CP-31398 affects gene expression and induces cell death both in a p53-dependent and independent manner. Thus, it appears that CP-3138 has other cellular targets than p53 that may account for its cellular toxicity.

Inventors of some embodiments of the invention have previously described the use of phage display libraries to select mutp53-reactivating peptides (PCT Publication No. WO2015/019318). Phage peptide display libraries have a much higher complexity than chemical libraries. The selection process was based on binding of peptides to an immobilized target, elution and amplification and finally identification by sequencing, enabling screening of high numbers of molecules in a short time. Different selection strategies were combined to select leads from different peptide libraries and deep sequencing of selected pools. Lead peptides were shown to endow mutp53 with WTp53-like activities in vitro and in live cells, and cause regression of mutp53-bearing tumors in several xenograft models. The lead peptide pCAP-250 having the sequence myr-RRHSTPHPD (SEQ ID NO: 24), was shown to bind the DNA Binding Domain (DBD) of p53. Binding of pCAP 250 and its peptide variants induces structural changes in the DBD, which directly influence the integrity and stability of the DBD-DNA binding interface region, namely the Helix-2 and the L1 loop structural motifs, which are essential for the ability of the DBD to bind the DNA. The binding of pCAP 250 and its peptide variants further affects additional residues at the surroundings of the helix 2 and the L1 loop structural motifs, creating a relatively large yet decisive affected patch on the DBD surface. These findings allowed the design of novel peptides that share the same interaction with the DBD of p53 and are able to at least partially reactivate a mutant p53 protein such peptides endowed with anti-cancer activity (PCT Publication No. WO2017/134671).

### SUMMARY OF THE INVENTION

The present invention provides peptide analogs that at least partially reactivate a mutant p53 protein (herein after “reactivating peptide analogs”). The invention is based in part on the finding that several modifications in the sequence of p53 reactivating peptides confer unexpected properties that render these peptide analogs to be more suitable as pharmaceutical agents, in particular for treatment of cancer. Peptide analogs according to the present invention are shown to have higher metabolic stability in plasma compared to similar peptides and higher in vivo inhibitory effect on tumor growth following bolus injection or continuous infusion.

The present invention provides, according to an aspect, a peptide analog is provided comprising the amino acid sequence Z₁-RRHSX₁X₂(Dab)PD-Z₂ (SEQ ID NO: 71) wherein:
X₁ is selected from D-Valine (v), L-Lysine (K) and D-Lysine (k);
X₂ is selected from the group consisting of L-Proline (P), D-Proline (p), di amino butyric acid (Dab), Di-methyl proline (Dmp), 4-tetrahydroisoquinoline-3-carboxylic acid (Tic), (S)-(−)-Indoline-2-carboxylic acid (Idc), Pipecolic acid (Pip), and octahydroindolecarboxylic acid (Oic);
Z₁ is a fatty acid residue comprising 16 to 19 carbon atoms; and
Z₂ denotes the carboxy terminus of the peptide analog which is: an unmodified C-terminus; an amidated C-terminus, connected to a side chain of an amino acid residue to form a cyclic peptide; or connected to a targeting moiety.

According to some embodiments, X₁X₂ is selected from the group consisting of: vP, vp, KP, kp, v(Dmp), v(Idc), v(Pip), v(Oic), and v(Tic).

According to some embodiments, a peptide analog is provided comprising the amino acid sequence Z₁-RRHSX₁X₂(Dab)PD-Z₂ (SEQ ID NO: 72) wherein:
X₁ is selected from D-Valine (v), L-Lysine (K) and D-Lysine (k);
X₂ is selected from the group consisting of D-Proline (p), di amino butyric acid (Dab), Di-methyl proline (Dmp), 4-tetrahydroisoquinoline-3-carboxylic acid (Tic), (S)-(−)-Indoline-2-carboxylic acid (Idc), Pipecolic acid (Pip), and octahydroindolecarboxylic acid (Oic);
Z₁ is a fatty acid residue comprising 16 to 19 carbon atoms; and
Z₂ denotes the carboxy terminus of the peptide analog which is: an unmodified C-terminus; an amidated C-terminus, connected to a side chain of an amino acid residue to form a cyclic peptide; or connected to a targeting moiety.

According to some embodiments, X₁X₂ is selected from the group consisting of: vp, kp, v(Dmp), v(Idc), v(Pip), v(Oic), and v(Tic).

According to yet other embodiments, X₁X₂ is selected from the group consisting of: vp, K*P, kp, v(Dmp), v(Idc), v(Pip), v(Oic), and v(Tic), wherein * denotes cyclization of the free amine group of the side chain of the Lysine residue with the carboxy terminus group.

According to some embodiments, the peptide analog is up to 15, 14 or 13 amino acids long. According to some embodiments, the peptide analog is up to 12, 11 or 10 amino acids long. According to some embodiments, the peptide analog consists of 9 to 12 amino acid residues. According to some embodiments, the peptide analog consists of 9 to 12 amino acid residues and a fatty acid residue comprising at least 16 carbon atoms.

According to some embodiments, the peptide analog is an isolated peptide analog.

According to some embodiments, the peptide analog comprises a carboxy terminus modified by amidation or by cyclization. According to other embodiments, the peptide analog comprises a free carboxy terminus.

According to some embodiments, the peptide analog comprises a fatty acid residue of at least 16 carbon atoms and at least one modification selected from a cyclization and a conjugation of a targeting moiety.

According to some embodiments, the targeting moiety is PDGED (SEQ ID NO: 70) or DGEA (SEQ ID NO: 54) or a retro-inverso orientation thereof.

According to some embodiments of the invention, the targeting moiety is RGDX (SEQ ID NO: 47, wherein X is absent or is any amino acid residue) or a retro-inverso orientation thereof.

According to some embodiments, the peptide analog comprises a fatty acid residue of at least 16 carbon atoms connected to the amino terminus and a cyclization between an amino acid side chain and the carboxy terminus.

According to some embodiments, the cyclization is a side chain to terminal cyclization, e.g., connection of a free amine of a side chain of an amino acid residue with the carboxy terminus.

According to some embodiments, the peptide analog comprises a fatty acid residue of at least 16 carbon atoms connected to the amino terminus and a targeting moiety connected to the carboxy terminus.

According to some embodiments, X₁ is selected from K and k and the peptide analog is cyclized by connecting the carboxy terminus group with the free amine group of the side chain of the K or k residues.

According to some embodiments, Z₁ is a fatty acid residue selected from the group consisting of palmitoyl (C₁₆); phtanoyl (CH₃)₄); heptadecanoyl (C₁₇); stearoyl (C₁₈) and nonadecanoyl (C₁₉).

According to some embodiments, Z₁ is selected from palmitoyl (C₁₆) and stearoyl (C₁₈).

According to some embodiments, the peptide analog is selected from the group consisting of:

PCAP724
str-RRHSkp(Dab)PD
(SEQ ID NO: 22, Cyclized by
connecting the D-Lys side chain
to the carboxy terminus);
pCAP673
palm-RRHSvP(Dab)PD-NH₂;
(SEQ ID NO: 3)
PCAP674
str-RRHSvP(Dab)PD-NH₂;
(SEQ ID NO: 27)
pCAP708
str-RRHSvp(Dab)PD-NH₂;
(SEQ ID NO: 6)
pCAP720
str-RRHSvp(Dab)PDGEA;
(SEQ ID NO: 18)
pCAP721
str-RRHSvp(Dab)PdGR;
(SEQ ID NO: 19)
pCAP716
str-RRHSv(Dmp)(Dab)PD-NH₂;
(SEQ ID NO: 14)
pCAP709
str-RRHSv(Idc)(Dab)PD-NH₂;
(SEQ ID NO: 7)
pCAP710
str-RRHSv(Pip)(Dab)PD-NH₂;
(SEQ ID NO: 8)
pCAP711
str-RRHSv(Oic)(Dab)PD-NH₂;
(SEQ ID NO: 9)
pCAP712
str-RRHSv(Tic)(Dab)PD-NH₂;
(SEQ ID NO: 10)
and
pCAP713
str-RRHSKP(Dab)PD
(SEQ ID NO: 11, Cyclized by connecting
the Lys side chain to the carboxy terminus).

According to some embodiments, X₂ is selected from the group consisting of D-Proline (p), di amino butyric acid (Dab), Di-methyl proline (Dmp), 4-tetrahydroisoquinoline-3-carboxylic acid (Tic), (S)-(−)-Indoline-2-carboxylic acid (Idc), Pipecolic acid (Pip), and octahydroindolecarboxylic acid (Oic) and the peptide analog is selected from the group consisting of:

PCAP724 str-RRHSkp(Dab)PD
(SEQ ID NO: 22, Cyclized by
connecting the D-Lys side
chain to the carboxy terminus);
pCAP708
str-RRHSvp(Dab)PD-NH₂;
(SEQ ID NO: 6)
pCAP720
str-RRHSvp(Dab)PDGEA;
(SEQ ID NO: 18)
pCAP721
str-RRHSvp(Dab)PdGR;
(SEQ ID NO: 19)
pCAP716
str-RRHSv(Dmp)(Dab)PD-NH₂;
(SEQ ID NO: 14)
pCAP709
str-RRHSv(Idc)(Dab)PD-NH₂;
(SEQ ID NO: 7)
pCAP710
str-RRHSv(Pip)(Dab)PD-NH₂;
(SEQ ID NO: 8)
pCAP711
str-RRHSv(Oic)(Dab)PD-NH₂;
(SEQ ID NO: 9)
and
pCAP712
str-RRHSv(Tic)(Dab)PD-NH₂.
(SEQ ID NO: 10)

According to some embodiments, X₁ is v, X₂ is selected from Dmp, Idc, Pip, Oic and Tic and the peptide analog is selected from the group consisting of:

PCAP716
(SEQ ID NO: 14)
str-RRHSv(Dmp)(Dab)PD-NH₂;
pCAP709
(SEQ ID NO: 7)
str-RRHSv(Idc)(Dab)PD-NH₂;
pCAP710
(SEQ ID NO: 8)
str-RRHSv(Pip)(Dab)PD-NH₂;
pCAP711
(SEQ ID NO: 9)
str-RRHSv(Oic)(Dab)PD-NH₂;
and
pCAP712
(SEQ ID NO: 10)
str-RRHSv(Tic)(Dab)PD-NH₂.

According to some embodiments, X₁ is K or k and X₂ is p.

According to some embodiments, the peptide analog is pCAP724, having the sequence str-RRHSkp(Dab)PD (SEQ ID NO: 22, cyclized by connecting the D-Lys side chain to the carboxy terminus).

The present invention also provides according to yet another aspect, a cyclic peptide analog comprising the sequence Z₁RRHSX₁X₂(Dab)PD (SEQ ID NO: 73), wherein X₁ is selected from D-Lysine (k) and L-Lysine (K), X₂ is selected from D-Proline (p) and L-Proline (P), Z₁ is a fatty acid residue comprising at least 16 carbon atoms, and the epsilon amine of the side chain of the Lysine or D-Lysine residue is connected to the carboxy terminus to form a cyclic peptide analog. According to some embodiments, the cyclic peptide analog is selected from SEQ ID NO: 22 and SEQ ID NO: 11. According to other embodiments, the cyclic peptide analog is set forth in SEQ ID NO: 22.

According to an aspect of some embodiments of the present invention there is provided an isolated peptide analog comprising an amino acid sequence as set forth in SEQ ID NO: 1 (RRHSX₁X₂X₃PD), wherein:
X₁ is any amino acid;
X₂ is selected from the group consisting of L-Proline (P), D-Proline (p), di amino butyric acid (Dab), Di-methyl proline (Dmp), 4-tetrahydroisoquinoline-3-carboxylic acid (Tic), (S)-(−)-Indoline-2-carboxylic acid (Idc), Pipecolic acid (Pip), and octahydroindolecarboxylic acid (Oic);
X₃ is Dab,
wherein the peptide analog is modified to include at least one of:
   (i) a hydrophobic moiety selected from the group consisting of palmitoyl (C₁₆); phtanoyl (CH₃)₄); heptadecanoyl (C₁₇); stearoyl (C₁₈) and nonadecanoyl (C₁₉);
   (ii) cyclization; and
   (iii) a targeting moiety;
wherein the peptide analog at least partially reactivates a mutant p53 protein.

According to some embodiments, X₁X₂ is selected from the group consisting of: vP, vp, KP, kp, v(Dmp), v(Idc), v(Pip), v(Oic), and v(Tic).

According to some embodiments, X₁X₂ is selected from the group consisting of: vp, kp, v(Dmp), v(Idc), v(Pip), v(Oic), and v(Tic).

According to yet other embodiments, X₁-X₂ is selected from the group consisting of: vp, K*P, kp, v(Dmp), v(Idc), v(Pip), v(Oic), and v(Tic), wherein * denotes cyclization of the free amine group of the side chain of the Lysine residue with the carboxy terminus group.

According to some embodiments, X₂ is selected from the group consisting of D-Proline (p), di amino butyric acid (Dab), Di-methyl proline (Dmp), 4-tetrahydroisoquinoline-3-carboxylic acid (Tic), (S)-(−)-Indoline-2-carboxylic acid (Idc), Pipecolic acid (Pip), and octahydroindolecarboxylic acid (Oic);

According to some embodiments, X₁X₂ is selected from KP and kp and the peptide analog is cyclized by connecting the carboxy terminus group with the free amine group of the side chain of the L-Lysine (K) or D-Lysine (k) residue.

According to some embodiments of the invention, the hydrophobic moiety is attached to an N-terminus of the peptide.

According to some embodiments of the invention, X₁ is selected from D-Lys (k), L-Lys (K), D-Val (v) and L-Val (V).

According to some embodiments of the invention, the targeting moiety is attached to a C-terminus of the peptide analog.

According to some embodiments of the invention, the targeting moiety is PDGED (SEQ ID NO: 70) or DGEA (SEQ ID NO: 54) or a retro-inverso orientation thereof.

According to some embodiments of the invention, the targeting moiety is RGDX (SEQ ID NO: 47, wherein X is absent or is any amino acid residue) or a retro-inverso orientation thereof.

According to some embodiments of the invention, X₁ is D-Lys (k) or L-Lys (K) and the cyclization is between the free epsilon amine of the side chain of the L-Lysine or D-Lysine residue and the C-terminus of the peptide analog.

According to some embodiments of the invention, the peptide is up to 15 amino acids long. According to some embodiments of the invention, the peptide is up to 10 amino acids long. According to some embodiments of the invention, the peptide consists of 9 to 12 amino acid residues. According to some embodiments of the invention, the peptide consists of 9 to 12 amino acid residues and a fatty acid residue comprising at least 16 carbon atoms.

According to some embodiments of the invention, the peptide analog comprises (i)+(ii).

According to some embodiments of the invention, the peptide analog comprises (i)+(iii).

According to some embodiments of the invention, the peptide analog comprises (ii)+(iii).

According to some embodiments of the invention, the peptide analog comprises (i)+(ii)+(iii).

According to some embodiments of the invention, the peptide is selected from the group consisting of SEQ ID NO: 22 (pCAP-724), SEQ ID NO: 11 (pCAP-713), SEQ ID NO: 14 (pCAP-716), SEQ ID NO: 15 (pCAP-717), SEQ ID NO: 17 (pCAP-719), SEQ ID NO: 6 (pCAP-708), and SEQ ID NO: 19 (pCAP-721).

According to some embodiments of the invention, the peptide is as set forth in SEQ ID NO: 22 (pCAP-724).

According to some embodiments of any aspect of the present invention, the peptide analog has an IC50 below 40 μM for inhibition of cell viability.

According to some embodiments of any aspect of the present invention, the peptide analog at least partially changes the conformation of the mutant p53 protein to a conformation of a wild-type (WT) p53 protein.

According to some embodiments of any aspect of the present invention, the peptide analog at least partially changes the conformation of the mutant p53 protein such that the mutant p53 protein is recognized by a monoclonal antibody directed against a WT p53 protein.

According to some embodiments any aspect of the present, the mutant p53 protein is not recognized, prior to treatment with a peptide analog according to the present invention, by a monoclonal antibody directed against a WT p53 protein.

According to some embodiments of any aspect of the present invention, the mutant p53 protein, upon binding to the peptide analog, is recognized by a monoclonal antibody directed against a WT p53 protein.

According to some embodiments of any aspect of the present invention, the peptide analog at least partially restores an activity of the mutant p53 protein to the activity of a WT p53 protein.

According to some embodiments of any aspect of the present invention, the activity is reducing viability of cells expressing the mutant p53 protein.

According to some embodiments of any aspect of the present invention, the activity is promoting apoptosis of cells expressing the mutant p53 protein.

According to some embodiments of any aspect of the present invention, the activity is binding to a p53 consensus DNA binding element in cells expressing the mutant p53 protein.

According to an aspect, the present invention provides an isolated peptide or peptide analog described above, for use in treating disease, disorder or condition associated with a mutant p53 protein.

According to another aspect, the present invention provides a pharmaceutical composition comprising at least one isolated peptide or peptide analog described above and a pharmaceutically acceptable excipient, diluent or carrier.

Pharmaceutical compositions according to the present invention may be formulated for any administration mode or route. According to some embodiments, the pharmaceutical composition is formulated for an administration route selected from injection and infusion.

According to some embodiments, the pharmaceutical composition is formulated for infusion by pump or for slow, sustained or delayed release.

According to yet another aspect, the present invention provides a method of treating a disease, disorder or condition associated with a mutant p53 protein, comprising administering to a subject in need thereof a therapeutically effective amount of a peptide analog described above or a pharmaceutical composition comprising it, thereby treating the disease, disorder or condition.

According to some embodiments of the invention, the disease is cancer.

According to some embodiments, the cancer is selected from the group consisting of: breast cancer, colon cancer, ovarian cancer and lung cancer.

In some embodiments, the cancer is a metastatic cancer.

In some embodiments, the cancer is a metastatic breast cancer, metastatic colon cancer, metastatic ovarian cancer or metastatic lung cancer.

Peptide analogs and pharmaceutical compositions comprising them may be administered according to the method of the present invention, using any suitable administration route.

According to some embodiments, the administration route is selected from parenteral, oral, topical, and transdermal administration. According to some embodiments the route of administration is via parenteral injection. According to some embodiments, the peptide analogs of the present invention are administered systemically, by parenteral routes, such as, intravenous (i.v.), subcutaneous (s.c.), intramuscular (i.m.), intraperitoneal (i.p.), or intranasal routes.

According to a specific embodiment, the route of administration is selected from injection and infusion.

Alternatively, or additionally, according to a specific embodiment, administering comprises continuous infusion, e.g. by slow-release delivery systems, pumps, and other known delivery systems for continuous infusion.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the Drawings:

FIGS. 1A and 1B are graphs showing viability of cancer cell expressing wild-type p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. The calculated IC50 values of all tested compounds is >40 μM.

FIGS. 2A and 2B are graphs showing viability of cancer cell expressing wild-type p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. The calculated IC50 values of all tested compounds is >40 μM.

FIGS. 3A and 3B are graphs showing viability of cancer cell expressing wild-type p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. The calculated IC50 values of all tested compounds is >40 μM.

FIGS. 4A and 4B are graphs showing viability of cancer cell expressing wild-type p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. The calculated IC50 values of all tested compounds is >40 μM.

FIGS. 5A and 5B are graphs showing viability of cancer cell expressing wild-type p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 5A: the calculated IC50 values of the tested compounds are: pCAP-250=18.86 μM, pCAP-553=18.92 μM and pCAP-669=19.6 μM. FIG. 5B: the calculated IC50 values of the tested compounds are: pCAP-250=10.6 μM, pCAP-553=13.2 μM and pCAP-669=10.42 μM.

FIGS. 6A and 6B are graphs showing viability of cancer cell expressing wild-type p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. The calculated IC50 of all tested compounds is >40 μM.

FIGS. 7A and 7B are graphs showing viability of cancer cell expressing wild-type p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 7A: The calculated IC50 values of the tested compounds are: pCAP-250=7.46 μM, pCAP-553=7.08 μM and pCAP-669=7.48 μM. FIG. 7B: The calculated IC50 values of the tested compounds are: pCAP-250=7.78 μM, pCAP-553=7.71 μM and pCAP-669=6.3 μM.

FIGS. 8A and 8B are graphs showing viability of cancer cell expressing mutant p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 8A: The calculated IC50 values of the tested compounds are: pCAP-250=0.47 μM, pCAP-553=0.45 μM and pCAP-669=0.57 μM. FIG. 8B: The calculated IC50 values of the tested compounds are: pCAP-250=0.75 μM, pCAP-553=0.78 μM and pCAP-669=0.69 μM.

FIGS. 9A and 9B are graphs showing viability of cancer cell expressing mutant p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 9A: The calculated IC50 values of the tested compounds are: pCAP-250=3.15 μM, pCAP-553=4.63 μM and pCAP-669=4.36 μM. FIG. 9B: The calculated IC50 values of the tested compounds are: pCAP-250=1.46 μM, pCAP-553=1.73 μM and pCAP-669=1.5 μM.

FIGS. 10A and 10B are graphs showing viability of cancer cell expressing mutant p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 10A: The calculated IC50 values of the tested compounds are: pCAP-250=2.29 μM, pCAP-553=1.81 μM and pCAP-669=2.6 μM. FIG. 10B: The calculated IC50 values of the tested compounds are: pCAP-250=0.8 μM, pCAP-553=1.6 μM and pCAP-669=1.62 μM.

FIGS. 11A and 11B are graphs showing viability of cancer cell expressing mutant p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 11A: The calculated IC50 values of the tested compounds are: pCAP-250=0.36 μM, pCAP-553=0.4 μM and pCAP-669=0.345 μM. FIG. 11B: The calculated IC50 values of the tested compounds are: pCAP-250=0.63 μM, pCAP-553=0.72 μM and pCAP-669=0.21 μM.

FIGS. 12A and 12B are graphs showing viability of cancer cell expressing mutant p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 12A: The calculated IC50 values of the tested compounds are: pCAP-250=3.6 μM, pCAP-553=3.73 μM and pCAP-669=3.66 μM. FIG. 12B: The calculated IC50 values of the tested compounds are: pCAP-250=0.68 μM, pCAP-553=0.73 μM and pCAP-669=0.7 μM.

FIGS. 13A and 13B are graphs showing viability of cancer cell expressing mutant p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 13A: The calculated IC50 values of the tested compounds are: pCAP-250=3.69 μM, pCAP-553=2.93 μM and pCAP-669=3.67 μM. FIG. 13B: The calculated IC50 values of the tested compounds are: pCAP-250=1.74 μM, pCAP-553=2.09 μM and pCAP-669=1.67 μM.

FIGS. 14A and 14B are graphs showing viability of cancer cell expressing mutant p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 14A: The calculated IC50 values of the tested compounds are: pCAP-250=1.5 μM, pCAP-553=1.63 μM and pCAP-669=1.8 μM. FIG. 14B: The calculated IC50 values of the tested compounds are: pCAP-250=1.17 μM, pCAP-553=1.62 μM and pCAP-669=1.42 μM.

FIGS. 15A and 15B are graphs showing viability of cancer cell expressing mutant p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 15A: The calculated IC50 values of the tested compounds are: pCAP-250=0.69 μM, pCAP-553=0.69 μM and pCAP-669=0.84 μM. FIG. 15B: The calculated IC50 values of the tested compounds are: pCAP-250=0.708 μM, pCAP-553=0.72 μM and pCAP-669=0.706 μM.

FIGS. 16A and 16B are graphs showing viability of cancer cell expressing mutant p53 in the presence of pCAP-250 (SEQ ID NO: 24), pCAP-553 (SEQ ID NO: 2), pCAP-669 (SEQ ID NO: 25), pCAP-704 (SEQ ID NO: 26) or cis-platinum. FIG. 16A: The calculated IC50 values of the tested compounds are: pCAP-250=2.84 μM, pCAP-553=2.73 μM and pCAP-669=4.201 FIG. 16B: The calculated IC50 values of the tested compounds are: pCAP-250=0.575 μM, pCAP-553=0.575 μM and pCAP-669=0.574 μM.

FIG. 17 is a graph showing the effect of fatty acid length on the activity of the peptide.

FIGS. 18A-18D are graphic presentation showing the effect of peptide modifications on cell viability of different mutant p53-expressing cancer cells. FIG. 18A: RXF-393 (renal carcinoma) cells, p53 hotspot mutation-R175H. FIG. 18B: SW-480 (colon carcinoma) cells, p53 hotspot mutation-R273H. FIG. 18C: WI38 (non-transformed fibroblasts) cells. FIG. 18D: PC9 (lung carcinoma) cells, p53 hotspot mutation-R248Q.

FIGS. 19A-19D are graphic presentation showing the effect of peptide modifications on cell viability of different mutant p53-expressing cancer cells. FIG. 19A: RXF-393 (renal carcinoma) cells, p53 hotspot mutation-R175H. FIG. 19B: PANC1 (pancreatic carcinoma) cells, p53 hotspot mutation-R273H. FIG. 19C: WI38 (non-transformed fibroblasts) cells. FIG. 19D: PC9 (lung carcinoma) cells, p53 hotspot mutation-R248Q.

FIGS. 20A-20D are graphic presentation showing the effect of peptide modifications on cell viability of different mutant p53-expressing cancer cells. FIG. 20A: RXF-393 (renal carcinoma) cells, p53 hotspot mutation-R175H. FIG. 20B: MIA PaCa-2 (pancreatic carcinoma) cells, p53 hotspot mutation-R248W. FIG. 20C: SW-480 (colon carcinoma) cells, p53 hotspot mutation-R273H. FIG. 20D: PC9 (lung carcinoma) cells, p53 hotspot mutation-R248Q.

FIG. 21 depicts PK profile for pCAP-724 (SEQ ID NO:22). LC-MS analysis of peptide concentration in the plasma of injected mice. Each point represents an average reading from 3 mice. Shown are mean plasma concentrations (mpk) of the peptide, following administration either intravenously at 1 mg/kg (IV) or subcutaneously at 5 mg/kg (SC).

FIGS. 22A-22C demonstrate the effect of pCAP-724 (SEQ ID NO: 22), on tumor growth in vivo. RXF-373 is a human renal cell carcinoma (RCC) cell line. RXF-373 cells, expressing endogenous mutant p53 and stably transduced to express luciferase, were injected into the hips of nude mice. When tumors reached visible size, mice were divided into the 5 indicated groups and treated accordingly. Shown is live imaging of each group of mice at the beginning of treatment and at termination of the experiment. FIG. 22A shows images of control mice subcutaneously injected with pCAP-722 twice a week, on days 21 and 42. FIG. 22B shows images of mice treated with subcutaneous injection of pCAP-724 twice a week, on days 21 and 42. FIG. 22C shows images of mice treated with a pump continuously administering pCAP-722, on days 24 and 39.

FIG. 23 is a logarithmic scale plot showing the luciferase readings of tumors as a function of time of treatment. Shown are averages before (until day 21) and after initiation of treatment. The background threshold detection level of the IVIS system in this experiment was about 5×10⁶ photons.

FIG. 24—Average tumor weight of the different groups of mice at the end of the experiment (day 42) described in FIG. 22.

### DESCRIPTION OF SPECIFIC EMBODIMENTS OF THE INVENTION

The present invention, in some embodiments thereof, relates to peptides and use of same in treating diseases, disorders or conditions associated with a mutant p53 protein.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have previously identified pCAP-553 myr-RRHSvP(L-Dab)PD (SEQ ID NO: 2, see PCT Publication No. WO2017/134671). This peptide was selected for further development since it has better solubility at physiological pH and has also shown improved efficacy relatively to pCAP-250, in viability assays with some of the tested cell lines. The present inventors have therefore taken further steps for improving the efficacy of the peptide in reducing viability of cancer cells. These steps included: fatty acid selection and positioning, substitution of residues including chirality, at positions 4 and 5 from the N-terminus, peptide cyclization, tumor targeting or combinations of same.

Thus, according to an aspect of the invention there is provided an isolated peptide comprising an amino acid sequence as set forth in SEQ ID NO: 1 (RRHSX₁X₂X₃PD), wherein:
X₁ is any amino acid;
X₂ is selected from the group consisting of D-Proline, Dab, L-Proline, Dmp, DMP and Tic;
X₃ is Dab,
wherein the peptide is modified to include at least one of:
   (i) a hydrophobic moiety selected from the group consisting of palmitoyl (C₁₆); phtanoyl ((CH₃)₄); heptadecanoyl (C₁₇); stearoyl (C₁₈) and nonadecanoyl (C₁₉);
   (ii) cyclization; and
   (ii) a targeting moiety;
wherein the peptide at least partially reactivates a mutant p53 protein.

The present invention also provides a peptide analog comprising the sequence Z₁-RRHSX₁X₂(Dab)PD-Z₂ (SEQ ID NO: 71) wherein:
X₁ is selected from L-Lysine (K) and D-Lysine (k);
X₂ is selected from the group consisting of L-Proline, D-Proline (p), di amino butyric acid (Dab), Di-methyl proline (Dmp), 4-tetrahydroisoquinoline-3-carboxylic acid (Tic), (S)-(−)-Indoline-2-carboxylic acid (Idc), Pipecolic acid (Pip), and octahydroindolecarboxylic acid (Oic);
Z₁ is a fatty acid residue comprising 16 to 19 carbon atoms; and
Z₂ denotes the carboxy terminus of the peptide analog which may be free, amidated, connected to a side chain of an amino acid residue to form a cyclic peptide, or connected to a targeting moiety.

Also provided according to the present invention is a peptide analog comprising the amino acid sequence Z₁-RRHSX₁X₂(Dab)PD-Z₂ (SEQ ID NO: 72) wherein:
X₁ is selected from D-Valine (v), L-Lysine (K) and D-Lysine (k);
X₂ is selected from the group consisting of D-Proline (p), di amino butyric acid (Dab), Di-methyl proline (Dmp), 4-tetrahydroisoquinoline-3-carboxylic acid (Tic), (S)-(−)-Indoline-2-carboxylic acid (Idc), Pipecolic acid (Pip), and octahydroindolecarboxylic acid (Oic);
Z₁ is a fatty acid residue comprising 16 to 19 carbon atoms; and
Z₂ denotes the carboxy terminus of the peptide analog which is: an unmodified C-terminus; an amidated C-terminus, connected to a side chain of an amino acid residue to form a cyclic peptide; or connected to a targeting moiety.

The present invention also provides cyclic peptide analogs comprising the sequence Z₁RRHSX₁p(Dab)PD (SEQ ID NO: 73), wherein X₁ is selected from D-Lysine (k) and L-Lysine (K), Z₁ is a fatty acid residue comprising at least 16 carbon atoms, and the epsilon amine of the side chain of the Lysine or D-Lysine residue is connected to the carboxy terminus to form a cyclic peptide analog.

As used herein the term “isolated” refers to at least partially separated from the natural environment e.g., from the body or from a peptide library.

As used herein the term “p53” also known as “TP53” refers to the gene sequence encoding the protein product of EC 2.7.1.37, generally functioning as a transcription factor, regulating the cell cycle, hence functioning, in its wild-type form, as a tumor suppressor gene. According to a specific embodiment, the p53 is a human p53.

As used herein, the terms “wild type p53”, “wt p53” and “WT p53” may interchangeably be used and are directed to a wild type p53 protein, having the conformation of a wild type p53 protein and hence, activity of a wild type p53 protein. In some embodiments, wild type p53 can be identified by a specific monoclonal antibody. In certain embodiments, the monoclonal antibody is PAb1620.

Structural data for the protein is available from PDBe RCSB.

The term “conformation” with respect to a protein is directed to the structural arrangement (folding) of a protein in space.

As used herein, the terms “mutant p53”, “Mut-p53”, “mutated p53”, and “p53 mutant” may interchangeably be used and are directed to a mutated p53 protein, incapable of efficiently functioning in a target cell. In some embodiments, a Mut-p53 cannot bind its target site. In some embodiments, a Mut-p53 is mutated at the DNA binding domain (DBD) region. In some embodiments, a Mut-p53 is misfolded in an inactive conformation. In some exemplary embodiments, the Mut-p53 is a temperature sensitive (ts) mut p53 R249S (R249S p53), a hot spot full length mutant p53 Mut-p53 R175H (R175H p53), or any other Mut-p53 protein. In some embodiments, a Mut-p53 is identified by a specific monoclonal antibody, capable of recognizing a misfolded conformation of p53 (induced by the mutation of the p53). In some embodiments, a Mut-p53 is identified by a specific monoclonal antibody. In certain embodiments, the monoclonal antibody is Ab420.

In certain embodiments, the mutant p53 protein comprises a mutation selected from the group consisting of R175H, V143A, R249S, R273H, R280K, P309S, P151S, P151H, C176S, C176F, H179L, Q192R, R213Q, Y220C, Y220D, R245S, R282W, D281G, S241F, C242R, R248Q, R248W, D281G, R273C and V274F. Each possibility represents a separate embodiment of the invention.

As referred to herein, the terms “reactivating peptide analog”, “reactivating peptide”, “Mut-p53 reactivating peptide”, “the peptide analog” or “the peptide” may interchangeably be used and are directed to a peptide capable of at least partially restoring activity to Mut-p53. The phrase “reactivating mutant p53 protein” as used herein refers to a peptide which upon its interaction with a mutant p53 protein, the mutant p53 protein increases at least one of its activities, wherein the activities are the activities of a wild type p53 protein. For example, upon its interaction with a peptide provided by the present invention, a mutant p53 protein may increase, directly or indirectly, the expression of pro-apoptotic proteins such as caspases in a cancer cell, in a similar way to what would a wild type p53 protein do in a similar situation or suppress tumors in vivo as can be assayed using a xenograft mouse model of the disease.

Without being bound by theory it is suggested that the reactivating peptide or peptide analog binds the mut p53 in the DBD and thermodynamically stabilizes the WTp53 protein folding and hence restore tumor suppression function.

In some embodiments, the reactivating peptide analogs can reactivate a Mut-p53 by affecting the conformation of the Mut-p53, to assume a conformation which is more similar to or identical to a native, WT p53. In some embodiments, the reactivating peptide analogs can reactivate a Mut-p53 to restore binding of the Mut-p53 to a WT p53 binding site in a target DNA. In some embodiments, the reactivating peptide analog can restore biochemical properties of the Mut-p53. In some embodiments, the reactivating peptide analog can induce the Mut-p53 protein to exhibit p53-selective inhibition of cancer cells. In some embodiments, the reactivating peptide analog can reactivate a Mut-p53 to have structural properties, biochemical properties, physiological properties and/or functional properties similar (i.e., ±, 10%, 20%, 30% difference between the Mut-p53 and WT p53) to or identical to a WT p53 protein such as determined in the binding/structural assays as described herein e.g., MST and NMR.

According to some embodiments of the invention the peptide analog is up to 10 amino acids long.

According to some embodiments of the invention the peptide analog is up to 15 amino acids long.

In some embodiments, the peptide analog is 9-30 amino acids in length. In some embodiments, the peptide analog is 10-30 amino acids in length. In some embodiments, the peptide analog is 12-30 amino acids in length. In some embodiments, the peptide analog is 3-25 amino acids in length. In some embodiments, the peptide analog is 9-25 amino acids in length. In some embodiments, the peptide analog is 12-25 amino acids in length. In some embodiments, the peptide analog is 9-22 amino acids in length. In some embodiments, the peptide analog is 9-20 amino acids in length. In some embodiments, the peptide analog is 9-18 amino acids in length. In some embodiments, the peptide analog is 9-16 amino acids in length. In some embodiments, the peptide analog is 9-14 amino acids in length. In some embodiments, the peptide analog is 9-12 amino acids in length. In some embodiments, the peptide analog is 12-22 amino acids in length. In some embodiments, the peptide analog is 9-10 amino acids in length.

The term “capable of at least partially reactivating a mutant p53 protein” or “at least partially reactivate a mutant p53 protein” as interchangeably used herein refers to a peptide or a peptide analog, wherein upon binding of the peptide to a mutant p53 protein, the mutant p53 protein gains or increases an activity similar to a corresponding activity of a wild type p53 protein.

According to some embodiments, the peptide or peptide analog comprises an amino acid sequence arranged in a space and configuration that allow interaction of the peptide or peptide analog with the DBD of p53 through at least one residue of the DNA Binding Domain (DBD) by which pCAP 250 (SEQ ID NO: 24) binds the DBD.

As used herein “the DNA Binding Domain” or “DBD” of p53 refers to the domain of p53 which binds a p53 responsive element in a target protein (e.g., a consensus DNA binding element comprises or consists the amino-acid sequence set forth in SEQ ID NO: 44 of WO2017/134671), typically attributed to residues 94-292, 91-292, 94-293, 94-296, 91-296, 91-293, 94-312 or 92-312 of human p53 (full length p53 GenBank: BAC16799.1, SEQ ID NO: 44 of WO2017/134671). According to a specific embodiment, the DBD is of a mutated p53.

Thus, a reactivating peptide analog according to some embodiments of the invention is typically associated with the DBD domain of p53 such that the reactive group(s) of the peptide or peptide analog are positioned in a sufficient proximity to corresponding reactive group(s) (typically side chains of amino acid residues) in the DBD, so as to allow the presence of an effective concentration of the peptide analog in the DBD and, in addition, the reactive groups of the peptide analog are positioned in a proper orientation, to allow overlap and thus a strong chemical interaction and low dissociation. A reactivating peptide or peptide analog, according to some embodiments of the invention therefore typically includes structural elements that are known to be involved in the interactions, and may also have a restriction of its conformational flexibility, so as to avoid conformational changes that would affect or weaken its association with DBD of p53.

According to some embodiments, the interaction is via Helix-2 and L1 of said DBD.

Typically, helix-2 is positioned between amino acids 276-289 and L1 is positioned between amino acids 112-124.

According to some embodiments, the interaction affects the structural stability of Helix-2 and/or L1 of said DBD, as assayed by NMR.

According to some embodiments, the at least one residue in the DBD by which the interaction with the peptide or peptide analog is mediated is selected from the group consisting of H115, G117 of L1 of the p53 and Y126 and V274 and G279 and R280 of the p53 (wt or mutant in which the difference in amino acids is typically of single amino acids that do not significantly affect amino acid numbering. However, the skilled artisan would know how to find the corresponding amino acid (in terms of composition and position in the mutant p53).

According to some embodiments the interaction of the peptide or peptide analog with the DBD is non-covalent, e.g., water-mediated hydrogen bonding interactions.

According to some embodiments the interaction is by at least one amino acid of the amino acid sequence.

According to some embodiments the interaction is by at least two amino acids of the amino acid sequence.

According to some embodiments the interaction is by at least three amino acids of the amino acid sequence.

According to some embodiments the interaction is by at least four amino acids of the amino acid sequence.

According to a specific embodiment, the interaction is to amino acid Trp146 and/or Gln144 of human p53. This interaction is probably via the Ser of the pCAP 250 or its likes in analogous structures as further described hereinbelow.

According to a specific embodiment, the interaction is to amino acid Tyr126, Asn128 and/or Asp268 of human p53.

According to another specific embodiment, the interaction is to amino acid Lys101 of human p53 via Asp10 of the pCAP 250 or its likes in analogous structures as further described hereinbelow.

According to another specific embodiment, the interaction is to amino acid Thr102 of human p53 via Asp10 of the pCAP 250 or its likes in analogous structures as further described hereinbelow.

According to another specific embodiment, the interaction is to amino acid Phe113 of human p53 via Thr6 of the pCAP 250 or its likes in analogous structures as further described hereinbelow.

According to another specific embodiment, the interaction is to amino acid Trp146 of human p53 via Ser5 of the pCAP 250 or its likes in analogous structures as further described hereinbelow.

According to another specific embodiment, the interaction is to amino acid Ser5 of human p53 via Thr6 of the pCAP 250 or its likes in analogous structures as further described hereinbelow.

According to another specific embodiment, the interaction is to amino acid His 8 of human p53 via Thr6 of the pCAP 250 or its likes in analogous structures as further described hereinbelow.

According to another specific embodiment, the interaction is to amino acid Glyl12 of human p53 via Ser5 of the pCAP 250 or its likes in analogous structures as further described hereinbelow.

According to another specific embodiment, the interaction is to amino acid Glyl12 of human p53 via Thr6 of the pCAP 250 or its likes in analogous structures as further described hereinbelow.

Methods of elucidating the amino acids either in the peptide or in the DBD which are critical for the interaction are well known in the art and include, but are not limited to crystallography, as well as the use of computer-based algorithms e.g., AnchorDock (Ben Shimon Structure. 2015 May 5; 23(5):929-40), Virtual crystallographic Calculators V.2. and the like.

According to a specific embodiment, the peptide analog comprises a consensus motif set forth in SEQ ID NO: 1 (RRHSX₁X₂X₃PD), wherein:
X₁ is any amino acid (see below for examples), e.g., Tables 1 and 2;
X₂ is selected from the group consisting of D-Proline, Dab, L-Proline, Dmp, DMPand tic;
X₃ is Dab,

According to a specific embodiment, X₁ is D-Lys (k), L-Lys (K), D-Val (v) or L-Val (V).

As used herein Dab is Diaminobutyric acid and Dmp is Di-methyl proline.

According to a specific embodiment the peptide is modified to include at least one of:
(i) a hydrophobic moiety selected from the group consisting of palmitoyl (C₁₆); phtanoyl ((CH₃)₄); heptadecanoyl (C₁₇); stearoyl (C₁₈) and nonadecanoyl (C₁₉);
(ii) cyclization; and
(ii) a targeting moiety;

According to a specific embodiment the peptide analog comprises (i)+(ii).

According to a specific embodiment the peptide analog comprises (i)+(iii).

According to a specific embodiment the peptide analog comprises (ii)+(iii).

According to a specific embodiment the peptide analog comprises (i)+(ii)+(iii).

The hydrophobic moiety is included in order to enhance the permeability through biological membranes e.g., cell membranes. The hydrophobic moiety according to the invention is preferably a lipid moiety, e.g., a fatty acid.

According to a specific embodiment the hydrophobic moiety is selected from the group consisting of palmitoyl (C₁₆); phtanoyl ((CH₃)₄); heptadecanoyl (C₁₈); stearoyl (C₁₈) and nonadecanoyl (C₁₉).

According to a specific embodiment the hydrophobic moiety is selected from the group consisting of palmitoyl (C₁₆) and stearoyl (C₁₈).

According to a specific embodiment, the hydrophobic moiety is attached to an N-terminus of the peptide.

N-terminal modification with a fatty acid can be done using methods which are well known in the art of chemistry. For example, palmitoylation or stearoylation can be done by covalently attaching a palmitoyl group or a stearoyl group via an amide bond to the alpha-amino group of an N-terminal amino acid of the peptide.

According to an additional or alternative embodiments, the peptide analog comprises a targeting moiety is attached to a C-terminus of the peptide.

As used herein “a targeting moiety” refers to a chemical moiety which can be for example a small molecule or a peptide or a combination of same that imparts the peptide with selectivity or specificity in binding to a target cell of interest.

Following are some examples of peptide targeting moieties that can be used according to some embodiments of the present invention.

The suggested functions of some targeting peptides are:

RGDX (SEQ ID NO: 47, wherein X is absent or is any amino acid residue), GRGDS (SEQ ID NO: 48), and RGDS (SEQ ID NO: 49) enhancement of bone and/or cartilage tissue formation; regulation of neurite outgrowth; promotion of myoblast adhesion, proliferation and/or differentiation; enhancement of endothelial cell adhesion and/or proliferation; Peptide KQAGDV (SEQ ID NO: 50) smooth muscle cell adhesion; YIGSR (SEQ ID NO: 51) Cell adhesion; REDV (SEQ ID NO: 52) Endothelial cell adhesion; GTPGPQGIAGQRGVV (SEQ ID NO: 53) Cell adhesion (osteoblasts) (P-15); PDGEA (SEQ ID NO: 70) Cell adhesion (osteoblasts); IKVAV (SEQ ID NO: 55) Neurite extension; RNIAEIIKDI (SEQ ID NO: 56) Neurite extension; KHIFSDDSSE (SEQ ID NO: 57) Astrocyte adhesion; VPGIG (SEQ ID NO: 58) Enhance elastic modulus of artificial extra cellular matrix (ECM); FHRRIKA (SEQ ID NO: 59) Improve osteoblastic mineralization; KRSR (SEQ ID NO: 60) Osteoblast adhesion; KFAKLAARLYRKA (SEQ ID NO: 61) Enhance neurite extension; KHKGRDVILKKDVR (SEQ ID NO: 62) Enhance neurite extension; YKKIIKKL (SEQ ID NO: 63) Enhance neurite extension; NSPVNSKIPKACCVPTELS (SEQ ID NO: 64) Osteoinduction AI; APGL (SEQ ID NO: 65) Collagenase mediated degradation; VRNX (SEQ ID NO: 66, wherein X is absent or is any amino acid residue) Plasmin mediated degradation; AAAAAAAAA (SEQ ID NO: 67) Elastase mediated degradation; Acetyl-GCRDGPQ (SEQ ID NO: 68)—Encourage cell-mediated; GIWGQDRCG (SEQ ID NO: 69) proteolytic degradation, remodeling and/or bone regeneration (with RGD and BMP presentation in vivo).

Contemplated are retro, inverso or retro-inverso combinations of the targeting moiety.

According to a specific embodiment, the targeting moiety is PDGED (SEQ ID NO: 70) or DGEA (SEQ ID NO: 54) or a retro-inverso orientation thereof.

According to a specific embodiment, the targeting moiety is RGDX (SEQ ID NO: 47, wherein X is absent or is any amino acid residue) or a retro-inverso orientation thereof.

As mentioned, the peptide analog can be linear or cyclic.

According to a specific embodiment, the peptide analog is cyclic.

Cyclic peptides and peptide analogs can either be synthesized in a cyclic form or configured so as to assume a cyclic form under desired conditions (e.g., physiological conditions).

For example, a peptide analog according to the teachings of the present invention can include at least two cysteine residues flanking the core peptide sequence. In this case, cyclization can be generated via formation of S—S bonds between the two Cys residues. Side-chain to side chain cyclization can also be generated via formation of an interaction bond of the formula —(—CH2—)n—S—CH2—C—, wherein n=1 or 2, which is possible, for example, through incorporation of Cys or homoCys and reaction of its free SH group with, e.g., bromoacetylated Lys, Orn, Dab or Dap. Furthermore, cyclization can be obtained, for example, through amide bond formation (—CO—NH or —NH—CO bonds), e.g., by incorporating Glu, Asp, Lys, Orn, di-amino butyric (Dab) acid, and/or di-aminopropionic (Dap) acid at various positions in the chain. The amide bond can be formed between amine groups of e.g., Lys or Orn with a carboxylic group of the peptide's C-terminus or of natural or non-natural side chain of an amino acid; or between carboxylic groups of e.g., Glu, Asp, etc., with an amine group of the peptide's N-terminus or of natural or non-natural side chain of an amino acid. Backbone to backbone cyclization can also be obtained through incorporation of modified amino acids of the formulas H—N((CH₂)ₙ—COOH)—C(R)H—COOH or H—N((CH₂)n-COOH)—C(R)H—NH₂, wherein n=1-4, and further wherein R is any natural or non-natural side chain of an amino acid which can form a bond (e.g., amide bond) with compatible groups of the peptide's termini or natural or non-natural side chain of an amino acid residue in the peptide analog.

According to a specific embodiment, X₁ is D-Lys or L-Lys and the cyclization is between a side chain of said X₁ to a C-terminus of the peptide analog.

The term “peptide” as used herein encompasses native peptides (either degradation products, synthetically synthesized peptides or recombinant peptides) and the term “peptide analog” encompasses peptidomimetics (typically, synthetically synthesized peptides), as well as peptoids and semipeptoids, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH₃)—CO—), ester bonds (—C(═O)—O—), ketomethylene bonds (—CO—CH₂—), sulfinylmethylene bonds (—S(═O)—CH₂—), α-aza bonds (—NH—N(R)—CO—), wherein R is any alkyl (e.g., methyl), amine bonds (—CH₂—NH—), sulfide bonds (—CH₂—S—), ethylene bonds (—CH₂—CH₂—), hydroxyethylene bonds (—CH(OH)—CH₂—), thioamide bonds (—CS—NH—), olefinic double bonds (—CH═CH—), fluorinated olefinic double bonds (—CF═CH—), retro amide bonds (—NH—CO—), peptide derivatives (—N(R)—CH₂—CO—), wherein R is the “normal” side chain, naturally present on the carbon atom.

These modifications can occur at any of the bonds along the peptide chain and even at several (2-3) bonds at the same time.

“Conservative substitution” refers to the substitution of an amino acid in one class by an amino acid of the same class, where a class is defined by common physico-chemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gin, Glu); Class IV (His, Arg, Lys); Class V (He, Leu, Val, Met); and Class VI (Phe, Tyr, Trp). For example, substitution of an Asp for another Class III residue such as Asn, Gin, or Glu, is a conservative substitution.

Other classifications include positive amino acids (Arg, His, Lys), negative amino acids (Asp, Glu), polar uncharged (Ser, Thr, Asn, Gln), hydrophobic side chains (Ala, Val, Ile, Leu, Met, Phe, Tyr, Trp).

“Non-conservative substitution” refers to the substitution of an amino acid in one class with an amino acid from another class; for example, substitution of an Ala, a Class II residue, with a Class III residue such as Asp, Asn, Glu, or Gin.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr. Other synthetic options are listed hereinbelow in Table 2.

The peptides and peptide analogs of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodemosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | |
|---|---|---|
| Amino Acid | Abbreviation | One-letter Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| ornithine | Orn | Hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methyhsoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylomithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2-diphenyl | Nmbc | 1,2,3,4-tetrahydroisoquinoline- | Tic |
| ethylamino)cyclopropane | | 3-carboxylic acid | |
| phosphoserine | pSer | phosphothreonine | pThr |
| phosphotyrosine | pTyr | O-methyl-tyrosine | |
| 2-aminoadipic acid | | Hydroxylysine | |

TABLE 3
Exemplary peptide analogs which are contemplated according
to some embodiments of the invention

| SEQ | pCAP | | |
|---|---|---|---|
| ID No. | No. | Sequence | Comments |
| 29 | 656 | myr-RRHSEP(Dab)PDH | |
| 30 | 657 | myr-RRHSEP(Dab)PAH | |
| 31 | 658 | myr-RRHSv(Aib)(Dab)PD | |
| 32 | 659 | myr-RRHSvP(Dab)PD-NH₂ | |
| 33 | 660 | myr-RAHSvP(Dab)PD-NH₂ | |
| 34 | 661 | myr-RRASvP(Dab)PD-NH₂ | |
| 35 | 662 | myr-RRHAvP(Dab)PD-NH₂ | |
| 36 | 663 | myr-RRHSAP(Dab)PD-NH₂ | |
| 37 | 664 | myr-RRHSvA(Dab)PD-NH₂ | |
| 38 | 665 | myr-RRHSvPAPD-NH₂ | |
| 39 | 666 | myr-RRHSvP(Dab)AD-NH₂ | |
| 40 | 667 | myr-RRHSvP(Dab)PA-NH₂ | |
| 41 | 668 | myr-RRHStP(Dab)PD-NH₂ | |
| 25 | 669 | myr-RRHST(Aib)HAD-NH₂ | |
| 43 | 670 | myr-RRHSvP(Orn)PD-NH₂ | |
| 44 | 671 | myr-RRHSvPKPD-NH₂ | |
| 45 | 672 | myr-RRHSsP(Dab)PD-NH₂ | |
| 3 | 673 | pam-RRHSvP(Dab)PD-NH₂ | pam—Palmitic |
| 27 | 674 | str-RRHSvP(Dab)PD-NH₂ | str—Stearic |
| 46 | 675 | myr-RRHSK-(FITC) | fluorescently (FITC) labeled to the Lys |
| | | P(Dab)PD-NH₂ | side chain |
| 4 | 706 | arc-RRHSvP(Dab)PD-NH₂ | arc—Arachidic acid |
| 5 | 707 | Beh-RRHSvP(Dab)PD-NH₂ | Beh = Behenic acid |
| 6 | 708 | str-RRHSvp(Dab)PD-NH₂ | p—D-proline |
| 7 | 709 | str-RRHSv(Idc)(Dab)PD-NH₂ | (S)-(-)-Indoline-2-carboxylic acid |
| 8 | 710 | str-RRHSv(Pip)(Dab)PD-NH₂ | Pipecolic acid |
| 9 | 711 | str-RRHSv(Oic)(Dab)PD | Oic—octahydroindolecarboxylic acid |
| 10 | 712 | str-RRHSv(Tic)(Dab)PD-NH₂ | Tic—4-tetrahydroisoquinoline-3- |
| | | | carboxylic acid |
| 11 | 713 | str-RRHSKP(Dab)PD-Cyc | Cyclization of Lysine side chain to C |
| | | | terminal carboxyl |
| 12 | 714 | str-RRKSvP(Dab)PD-Cyc | Cyclization of Lysine side chain to C |
| | | | terminal carboxyl |
| 13 | 715 | str-RRKHSvP(Dab)PD-Cyc | Cyclization of Lysine side chain to C |
| | | | terminal carboxyl |
| 14 | 716 | str-RRHSv(Dmp)(Dab)PD-NH₂ | Dmp—Di-methyl proline |
| 15 | 717 | str-RRHSvP(Dab)PD-NH₂ | |
| 17 | 719 | str-RRHSk(tic)(Dab)PD-NH₂ | tic—D Tic |
| 18 | 720 | str-RRHSvp(Dab)PDGEA | C-terminal targeting sequence to tumor |
| | | | vasculature |
| 19 | 721 | str-RRHSvp(Dab)PdGR | C-terminal targeting sequence to tumor |
| | | | vasculature |
| 22 | 724 | str-RRHSkp(Dab)PD-Cyc | Cyclization of Lysine side chain to C |
| | | | terminal carboxyl |
| 28 | 729 | str-RRHS(Dab)p(Dab) | Cyclization of Dab side chain to C |
| | | PD-Cyc | terminal carboxyl |

Some control peptides and peptide analogs used in the examples below

| SEQ | pCAP | | |
|---|---|---|---|
| ID No. | No. | Sequence | |
| 26 | 704 | myr-DPHTS | Scrambled sequence- |
| | | PRHR | pCAP-250 control |
| 20 | 722 | str- | Scrambled sequence |
| | | DPHvSPR | pCAP-674 control |
| | | (Dab)R- | |
| | | NH₂ | |
| 21 | 723 | str- | Scrambled sequence |
| | | RDPHvSPR | pCAP-674 control |
| | | (Dab)-NH₂ | |
| 23 | 725 | str- | Scrambled sequence |
| | | DPHKSPR | pCAP-724 control |
| | | (Dab)R- | |
| | | Cyc | |

According to a specific embodiment, the peptide analog is selected from the group consisting of SEQ ID NO: 11 (pCAP-713), SEQ ID NO: 14 (pCAP-716), SEQ ID NO: 15 (pCAP-717/674), SEQ ID NO: 17 (pCAP-719) SEQ ID NO: 6 (pCAP-708), SEQ ID NO: 19 (pCAP-721) and SEQ ID NO: 22 (pCAP-724).

According to a specific embodiment, the peptide analog is as set forth in SEQ ID NO: 22 (pCAP-724).

In some embodiments, a peptide is chemically modified, such as described hereinabove to form a peptide analog.

“Chemically modified” refers to an amino acid that is modified either by natural processes, or by chemical modification techniques which are well known in the art. Among the numerous known modifications, typical, but not exclusive examples include: acetylation, acylation, amidation, ADP-ribosylation, glycosylation, glycosaminoglycanation, GPI anchor formation, covalent attachment of a lipid or lipid derivative, methylation, pegylation, prenylation, phosphorylation, ubiquitination, or any similar process (see e.g., SEQ ID NOs: 3-23).

According to a specific embodiment, the peptide analog comprises C-terminal amidation.

Yet alternatively or additionally the peptide analog is conjugated to non-proteinaceous non-toxic moiety such as, but are not limited to, polyethylene glycol (PEG), Polyvinyl pyrrolidone (PVP), poly(styrene comaleic anhydride) (SMA), and divinyl ether and maleic anhydride copolymer (DIVEMA).

The peptide analogs of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide analogs of some embodiments of the invention involves solid phase peptide synthesis.

Large scale peptide synthesis is described by Andersson Biopolymers 2000; 55(3):227-50.

In certain embodiments, the peptide analog at least partially changes the conformation of the mutant p53 protein to a conformation of a wild-type (WT) p53 protein.

Known in the art are antibodies that specifically recognize only wild type p53 proteins. Such antibodies are highly useful in determining whether a certain p53 protein, either wild type or mutant, holds the conformation of a wild type, functional p53 protein. Thus, in certain embodiments, the peptide analog at least partially changes the conformation of the mutant p53 protein such that the mutant p53 protein is recognized by a monoclonal antibody exclusively directed against a WT p53 protein or against a p53 protein holding a WT p53 protein conformation. In certain embodiments, the monoclonal antibody is PAb1620.

It should be understood that since p53 is expressed from both alleles, the overall content of intra-cellular p53 can be either wild-type (wt/wt), mixture of wt and mutant p53 (wt/mut) or mutant p53 only (when both alleles are mutated (mut/mut), or one allele is deleted (mut/−)). In cancer, the situation is often wt/mut, mut/mut or mut/−. Since p53 acts as a tetramer, mutant p53 proteins may abrogate the activity of wild type p53 proteins, which may exist in the cancer's cells. Therefore, the peptide analogs provided by the present invention are particularly useful in treating cancers in which increasing the level of wild type p53 proteins is not fruitful.

In certain embodiments, the peptide analog at least partially restores the activity of the mutant p53 protein to at least one of the activities of a WT p53 protein.

As used herein the term “reducing” refers to statistically significantly decreasing a certain phenotype by at least about 10%, 20%, 30%, 40%, 50%, 60%, 70%75%, 80%, 95% or even 100% as compared to a control (e.g., same cell/animal system treated with a control vehicle or non-treated at all) under the same assay conditions.

As used herein the term “increasing” or “improving” refers to statistically significantly increasing a certain phenotype by at least about 10%, 20%, 30%, 40%, 50%, 60%, 70%, 75%, 80%, 95% or even 100% as compared to a control (e.g., same cell/animal system treated with a control vehicle or non-treated at all) under the same assay conditions.

The term “cells expressing the mutant p53 protein” as used herein refers to cells which express from at least one allele a mutant p53 protein. In certain embodiments, the term “cells expressing the mutant p53 protein” is interchangeable with “cancer cells”.

The term “pro-apoptotic genes” refers to a gene, or a multitude of genes, involved in apoptosis, either directly (such as certain caspases) or indirectly (for example, as part of a signal transduction cascade).

In certain embodiments, the activity is reducing viability of cells expressing the mutant p53 protein. In certain embodiments, the activity is promoting apoptosis of cells expressing the mutant p53 protein. In certain embodiments, the activity is activating pro-apoptotic genes of cells expressing said mutant p53 protein. In certain embodiments, the pro-apoptotic genes are selected from the group consisting of CD95, Bax, DR4, DR5, PUMA, NOXA, Bid, 53AIP1 and PERP. Each possibility represents a separate embodiment of the invention.

In certain embodiments, the activity is binding to a p53 consensus DNA binding element in cells expressing the mutant p53 protein.

Methods of monitoring cellular changes induced by the any of the peptides of the present invention are known in the art and include for example, Crystal Violet Viability Assay (see Example 1), the MTT test which is based on the selective ability of living cells to reduce the yellow salt MTT (3-(4,5-dimethylthiazolyl-2)-2,5-diphenyltetrazolium bromide) (Sigma, Aldrich St Louis, Mo., USA) to a purple-blue insoluble formazan precipitate; the BrDu assay [Cell Proliferation ELISA BrdU colorimetric kit (Roche, Mannheim, Germany]; the TUNEL assay [Roche, Mannheim, Germany]; the Annexin V assay [ApoAlert® Annexin V Apoptosis Kit (Clontech Laboratories, Inc., CA, USA)]; the Senescence associated-β-galactosidase assay (Dimri G P, Lee X, et al. 1995. A biomarker that identifies senescent human cells in culture and in aging skin in vivo. Proc Natl Acad Sci USA 92:9363-9367); as well as various RNA and protein detection methods (which detect level of expression and/or activity) which are further described herein below.

In certain embodiments, the binding results in at least partial activation of an endogenous p53 target gene. In certain embodiments, the endogenous target gene is selected from the group consisting of p21, MDM2 and PUMA. Each possibility represents a separate embodiment of the invention.

In certain embodiments, the mutant p53 protein is of a different conformation than a WT p53 protein. In certain embodiments, the mutant p53 protein is at least partly inactive compared to a WT p53 protein.

In certain embodiments, the mutant p53 protein is not recognized by a monoclonal antibody directed against a WT p53 protein. In certain embodiments, the mutant p53 protein, upon binding to the peptide, is recognized by a monoclonal antibody directed against a WT p53 protein. In certain embodiments, the monoclonal antibody is PAb1620.

In some embodiments, the reactivating peptide analogs can reactivate a Mut-p53 to have structural properties, biochemical properties, physiological properties and/or functional properties similar to or identical to a WT p53 protein.

According to some embodiments, there are provided Mut-p53 reactivating peptide analogs, wherein the peptide analogs are in the length of about 3-25 amino acids. In some embodiments, the Mut-p53 reactivating peptide analogs are in the length of about 4-15 amino acids. In some embodiments, the Mut-p53 reactivating peptide analogs are in the length of about 7-12 amino acids. In some embodiments, the Mut-p53 reactivating peptide analogs are in the length of 7 amino acids. In some embodiments, the Mut-p53 reactivating peptide analogs are in the length of 12 amino acids. Each possibility represents a separate embodiment of the invention.

Other peptide lengths are recited throughout the application. Each possibility represents a separate embodiment of the invention.

According to some embodiments, a Mut-p53 reactivating peptide analog can affect Mut-p53 such that it can trans-activates a reporter gene (such as Luciferase) having WT p53 binding element in its promoter. In some embodiments the transactivation of the reporter gene may be performed in vitro (for example, in a test tube or well), or in-vivo in a cell, harboring the reporter gene construct.

According to some embodiments, a Mut-p53 reactivating peptide analog can bind to the DNA binding Domain (DBD) of a mutated p53. In some embodiments, the mutated p53 harbors a mutation in its DBD.

According to a specific embodiment, the peptide analog is endowed with clinically relevant IC50.

According to a specific embodiment, the IC50 is below 40 μM.

According to a specific embodiment, the IC50 is below 35 μM.

According to a specific embodiment, the IC50 is below 30 μM.

According to a specific embodiment, the IC50 is below 25 μM.

According to a specific embodiment, the IC50 is below 20 μM.

According to a specific embodiment, the IC50 is below 10 μM.

According to a specific embodiment, the IC50 is below 5 μM.

According to a specific embodiment, the IC50 is below 3 μM.

According to a specific embodiment, the IC50 is below 2 μM.

According to a specific embodiment, the IC50 is below 1 μM.

According to a specific embodiment, the IC50 is below 0.5 μM.

According to a specific embodiment, the IC50 is below 0.1 μM.

According to a specific embodiment, the IC50 is 0.01-0.1 μM.

The term “pharmaceutical composition” as used herein refers to any composition comprising at least one pharmaceutically active ingredient.

The term “associated with a mutant p53 protein” as used herein refers to any disease, disorder or condition which is caused by a mutant p53 protein or its progression relates to the presence of a mutant p53 protein in a cell or an organ.

It should be understood that since p53 is expressed from both alleles, the overall content of intra-cellular p53 can be either wild-type (wt/wt), mixture of wt and mutant p53 (wt/mut) or mutant p53 only (when both alleles are mutated (mut/mut), and one allele is deleted (mut/−)). In cancer, the situation is often wt/mut, mut/mut or mut/−. Since p53 acts as a tetramer, mutant p53 proteins may abrogate the activity of wild type p53 proteins, which do exist in the cancer's cells. Therefore, the peptide analogs provided by the present invention are particularly useful in treating cancers. Of note, the cell may have more than two p53 alleles at least one of which being of mutant p53.

The term “therapeutically effective amount” as used herein refers to an amount of a composition containing a peptide analog according to the present invention that is sufficient to reduce, decrease, and/or inhibit a disease, disorder or condition in an individual.

According to an aspect of the invention there is provided a method of treating a disease, disorder or condition associated with a mutant p53 protein, comprising administering to a subject in need thereof a therapeutically effective amount of the isolated peptide analog as described herein (e.g., SEQ ID NO: 22, 14, 15, 17 or 11), thereby treating said disease, disorder or condition.

According to an aspect of the invention there is provided a method of treating a disease, disorder or condition associated with a mutant p53 protein, comprising administering to a subject in need thereof a therapeutically effective amount of an isolated peptide analog as described herein, thereby treating said disease, disorder or condition.

According to a specific embodiment, the therapeutically effective amount is 0.01-50 mg/kg per day, 0.01-45 mg/kg per day, 0.01-40 mg/kg per day, 0.01-35 mg/kg per day, 0.01-30 mg/kg per day, 0.01-25 mg/kg per day, 0.01-20 mg/kg per day, 0.01-10 mg/kg per day, 0.1-50 mg/kg per day, 0.1-45 mg/kg per day, 0.1-40 mg/kg per day, 0.1-35 mg/kg per day, 0.1-30 mg/kg per day, 0.1-25 mg/kg per day, 0.1-20 mg/kg per day, 0.1-10 mg/kg per day, 1-50 mg/kg per day, 1-45 mg/kg per day, 1-40 mg/kg per day, 1-35 mg/kg per day, 1-30 mg/kg per day, 1-25 mg/kg per day, 1-20 mg/kg per day, 1-10 mg/kg per day, 10-50 mg/kg per day, 10-45 mg/kg per day, 10-40 mg/kg per day, 10-35 mg/kg per day, 10-30 mg/kg per day, 10-25 mg/kg per day, 10-20 mg/kg per day, 5-10 mg/kg per day, 1-5 mg/kg per day, 1-4.5 mg/kg per day, 1-4 mg/kg per day, 1-3.5 mg/kg per day, 1-3 mg/kg per day, 1-2.5 mg/kg per day, 1-2 mg/kg per day, 1-1.5 mg/kg per day, 0.1-3 mg/kg per day or 0.1-2 mg/kg per day 0.5-3 mg/kg per day or 0.5-2 mg/kg per day, 0.01-0.3 mg/kg per day or 0.01-0.2 mg/kg per day (e.g., 0.01-0.35 mg/kg per day, 0.01-0.35 mg/kg per day, 0.01-0.15 mg/kg per day, 0.01-0.1 mg/kg per day, 0.01-0.095 mg/kg per day, 0.01-0.09 mg/kg per day, 0.01-0.085 mg/kg per day, 0.01-0.08 mg/kg per day, 0.01-0.075 mg/kg per day, 0.01-0.07 mg/kg per day, 0.01-0.065 mg/kg per day, 0.01-0.06 mg/kg per day, 0.01-0.055 mg/kg per day, 0.01-0.05 mg/kg per day, 0.01-0.45 mg/kg per day, 0.01-0.04 mg/kg per day, 0.01-0.035 mg/kg per day, 0.01-0.03 mg/kg per day).

As referred to herein, the term “treating a disease” or “treating a condition” is directed to administering a composition, which includes at least one agent, effective to ameliorate symptoms associated with a disease, to lessen the severity or cure the disease, or to prevent the disease from occurring in a subject. Administration may include any administration route. In some embodiments, the disease is a disease that is caused by or related to the presence of a mutated p53 in a cell, tissue, organ, body, and the like. In some embodiments, the disease is cancer. In some embodiments, the cancer is selected from the group consisting of breast cancer, colon cancer, ovarian cancer and lung cancer.

In some embodiments, the cancer is a metastatic cancer.

In some embodiments, the cancer is a metastatic breast cancer, metastatic colon cancer, metastatic ovarian cancer or metastatic lung cancer.

Each possibility represents a separate embodiment of the invention. In some embodiments, the subject is a mammal, such as a human. In some embodiments, the subject is a mammal animal. In some embodiments, the subject is a non-mammal animal. In some embodiments the subject is diagnosed with the disease, condition or disorder.

In some embodiments, cancer is adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, ewings family of tumors (pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, small cell, lymphoma, AIDS-related, lymphoma, central nervous system (primary), lymphoma, cutaneous T-cell, lymphoma, hodgkin's disease, non-hodgkin's disease, malignant mesothelioma, melanoma, merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, sezary syndrome, skin cancer, cutaneous T-cell lymphoma, skin cancer, kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, or wilms' tumor.

In some embodiments, the cancer is a lung cancer.

In some embodiments, the cancer is an ovarian cancer.

In some embodiments, the cancer is a triple negative breast cancer.

In some embodiments, the cancer is a metastatic lung cancer.

In some embodiments, the cancer is a metastatic ovarian cancer.

In some embodiments, the cancer is a metastatic triple negative breast cancer.

In some embodiments, cancer is a non-solid tumor such as a blood cancer. In another embodiment, a non-solid tumor or blood cancer is leukemia or lymphoma. In another embodiment, a non-solid tumor or blood cancer is acute lymphoblastic leukemia (ALL). In another embodiment, a non-solid tumor or blood cancer is acute myelogenous leukemia (AML). In another embodiment, a non-solid tumor or blood cancer is chronic lymphocytic leukemia (CLL). In another embodiment, a non-solid tumor or blood cancer is small lymphocytic lymphoma (SLL). In another embodiment, a non-solid tumor or blood cancer is chronic myelogenous leukemia (CML). In another embodiment, a non-solid tumor or blood cancer is acute monocytic leukemia (AMOL). In another embodiment, a non-solid tumor or blood cancer is Hodgkin's lymphomas (any of the four subtypes). In another embodiment, a non-solid tumor or blood cancer is Non-Hodgkin's lymphomas (any of the subtypes). In another embodiment, a non-solid tumor or blood cancer is myeloid leukemia.

For use in the methods of the invention, the peptides and peptide analogs may be formulated in a conventional manner using one or more pharmaceutically acceptable carriers, stabilizers or excipients (vehicles) to form a pharmaceutical composition as is known in the art, in particular with respect to protein active agents. Carrier(s) are “acceptable” in the sense of being compatible with the other ingredients of the composition and not deleterious to the recipient thereof. Suitable carriers typically include physiological saline or ethanol polyols such as glycerol or propylene glycol.

The reactivating peptides and peptide analogs may be formulated as neutral or salt forms. Pharmaceutically acceptable salts include the acid addition salts (formed with free amino groups) and which are formed with inorganic acids such as hydrochloric or phosphoric acids, or such organic acids such as acetic, oxalic, tartaric and maleic. Salts formed with the free carboxyl groups may also be derived from inorganic bases such as sodium, potassium, ammonium, calcium, or ferric hydroxides, and organic bases as isopropylamine, trimethylamine, 2-ethylamino ethanol, histidine and procaine.

The compositions may be suitably formulated for intravenous, intramuscular, subcutaneous, or intraperitoneal administration and conveniently comprise sterile aqueous solutions of the reactivating peptide analogs, which are preferably isotonic with the blood of the recipient. Such formulations are typically prepared by dissolving solid active ingredient in water containing physiologically compatible substances such as sodium chloride, glycine, and the like, and having a buffered pH compatible with physiological conditions to produce an aqueous solution, and rendering said solution sterile. These may be prepared in unit or multi-dose containers, for example, sealed ampoules or vials.

The compositions may incorporate a stabilizer, such as for example polyethylene glycol, proteins, saccharides (for example trehalose), amino acids, inorganic acids and admixtures thereof. Stabilizers are used in aqueous solutions at the appropriate concentration and pH. The pH of the aqueous solution is adjusted to be within the range of 5.0-9.0, preferably within the range of 6-8. In formulating the reactivating peptide analogs, anti-adsorption agent may be used. Other suitable excipients may typically include an antioxidant such as ascorbic acid.

The compositions may be formulated as controlled release preparations which may be achieved through the use of polymer to complex or absorb the proteins. Appropriate polymers for controlled release formulations include for example polyester, polyamino acids, polyvinyl, pyrrolidone, ethylenevinylacetate, and methylcellulose. Another possible method for controlled release is to incorporate the reactivating peptide analogs into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatin-microcapsules and poly(methylmethacylate) microcapsules, respectively, or in colloidal drug delivery systems, for example, liposomes, albumin microspheres, microemulsions, nanoparticles, and nanocapsules or in macroemulsions.

In some embodiments, the reactivating peptide analogs of the invention may be formulated in peroral or oral compositions and in some embodiments, comprise liquid solutions, emulsions, suspensions, and the like. In some embodiments, pharmaceutically-acceptable carriers suitable for preparation of such compositions are well known in the art. In some embodiments, liquid oral compositions comprise from about 0.001% to about 0.9% of reactivating peptide analogs, or in another embodiment, from about 0.01% to about 10%.

In some embodiments, compositions for use in the methods of this invention comprise solutions or emulsions, which in some embodiments are aqueous solutions or emulsions comprising a safe and effective amount of a reactivating peptide analog and optionally, other compounds, intended for topical intranasal administration.

In some embodiments, injectable solutions of the invention are formulated in aqueous solutions. In one embodiment, injectable solutions of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

The reactivating peptide analogs of the invention may be administered by any suitable administration route, selected from oral, topical, transdermal or parenteral administration. According to some embodiments the route of administration is via topical application selected from dermal, vaginal, rectal, inhalation, intranasal, ocular, auricular and buccal. According to some embodiments the route of administration is via parenteral injection. In various embodiments, the step of administering is carried out by a parenteral route selected from the group consisting of intravenous, intramuscular, subcutaneous, intradermal, intraperitoneal, intraarterial, intracerebral, intracerebroventricular, intraosseous and intrathecal. For example, the reactivating peptide analogs may be administered systemically, for example, by parenteral routes, such as, intraperitoneal (i.p.), intravenous (i.v.), subcutaneous, or intramuscular routes. The reactivating peptide analogs of the invention and/or any optional additional agent may be administered systemically, for example, by intranasal administration. The reactivating peptide analogs of the invention and/or any optional additional agent may be administered systemically, for example, by oral administration, by using specific compositions or formulations capable of providing oral bioavailability to proteins. The reactivating peptide analogs of the invention and/or any optional additional agent may be administered locally.

According to a specific embodiment, administering comprises subcutaneous administering.

Alternatively, or additionally, according to a specific embodiment, administering comprises continuous infusion.

Thus the reactivating peptide analogs of the present invention can also be delivered by slow-release delivery systems, pumps, and other known delivery systems for continuous infusion for example in the following doses e.g., 0.01-0.3 mg/kg per day, 0.01-0.15 mg/kg per day, 0.01-0.1 mg/kg per day, 0.01-0.095 mg/kg per day, 0.01-0.09 mg/kg per day, 0.01-0.085 mg/kg per day, 0.01-0.08 mg/kg per day, 0.01-0.075 mg/kg per day, 0.01-0.07 mg/kg per day, 0.01-0.065 mg/kg per day, 0.01-0.06 mg/kg per day, 0.01-0.055 mg/kg per day, 0.01-0.05 mg/kg per day, 0.01-0.45 mg/kg per day, 0.01-0.04 mg/kg per day, 0.01-0.035 mg/kg per day, 0.01-0.03 mg/kg per day). Dosing regimens may be varied to provide the desired circulating levels of particular reactivating peptide analogs based on its pharmacokinetics. Thus, doses are calculated so that the desired circulating level of therapeutic agent is maintained.

Typically, the effective dose is determined by the activity of the reactivating peptide analogs and the condition of the subject, as well as the body weight or surface area of the subject to be treated. The size of the dose and the dosing regime is also determined by the existence, nature, and extent of any adverse side effects that accompany the administration of the reactivating peptide analogs in the particular subject.

In some embodiments, there is provided a kit for treating or preventing a p53 related condition. In some embodiments, the kit comprises a container (such as a vial) comprising a Mut-p53 reactivating peptide analog in a suitable buffer and instructions for use for administration of the reactivating peptide analog.

It is suggested that the efficacy of treatment with the peptides of the invention may be augmented when combined with gold standard treatments (e.g., anti-cancer therapy). Thus, the peptide can be used to treat diseases or conditions associated with p53 (as described hereinabove) alone or in combination with other established or experimental therapeutic regimen for such disorders. It will be appreciated that treatment with additional therapeutic methods or compositions has the potential to significantly reduce the effective clinical doses of such treatments, thereby reducing the often devastating negative side effects and high cost of the treatment.

Therapeutic regimen for treatment of cancer suitable for combination with the peptide analogs of some embodiments of the invention include, but are not limited to chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy. According to a specific embodiment, the chemotherapy is platinum-based.

Anti-Cancer Drugs

Anti-cancer drugs that can be co-administered with the compounds of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleuro sine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Specific examples of platinum-based chemotherapies include, but are not limited to, cisplatin, the first to be developed, carboplatin, a second-generation platinum-based antineoplastic agent, oxaliplatin, satraplatin, picoplatin, Nedaplatin, Triplatin, Lipoplatin, a liposomal version of cisplatin.

Kits and articles or manufacture for effecting combination treatments as described herein (e.g., the peptide together with platinum-based chemotherapy) are also contemplated herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences including minor sequence variations, resulting from, e.g., sequencing errors.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Experimental Procedures

Crystal Violet Viability Assay

Cells were cultured in 96 wells plates with 2500-4000 cells/well. Serial dilutions of different peptides were added and the plates incubated for additional 48 h at 37° C. Then medium was removed and cell viability was determined by staining the cells with crystal violet (0.05%) in methanol/PBS (1:5, v/v), for 10 min, followed by 3 washes with PBS. 10% acetic acid was added to each well for 10 min. OD was determined at 595 nm.

Peptide Synthesis

Peptides were purchased from DGpeptides, FeiJiaTang Road 588, Downtown, Hangzhou city, Zhejiang province, China.

### Example 2

The Effect of pCAP 250 and its Derivatives is Cancer-Specific

In an effort to develop peptides towards clinical applications, the effect of various peptide analogs was examined on a large array of cancer cell lines. Several non-transformed cell lines were tested as well, in order to evaluate the toxicity of the peptides in normal tissues and to get an estimate of the therapeutic window of the peptides.

Viability assays were performed with 3 peptides: pCAP-250 (SEQ ID NO: 24), pCAP-553 (SEQ ID NO: 2) and pCAP-669 (SEQ ID NO: 25), while cis-platinum served as a cytotoxic agent control. Two different methods: (1) crystal violet, which stains cells that adhere to the culture dish and are expected to be viable, as opposed to dead cells that detach from the dish, and (2) CellTiterGlo (CTG), which measures a fluorescent signal proportional to the amount of ATP and therefore to the number of viable cells. Although there was some variability in IC50 values between the two assays, this variability does not exceed the variability that is typically observed between two independent experiments performed using the same assay.

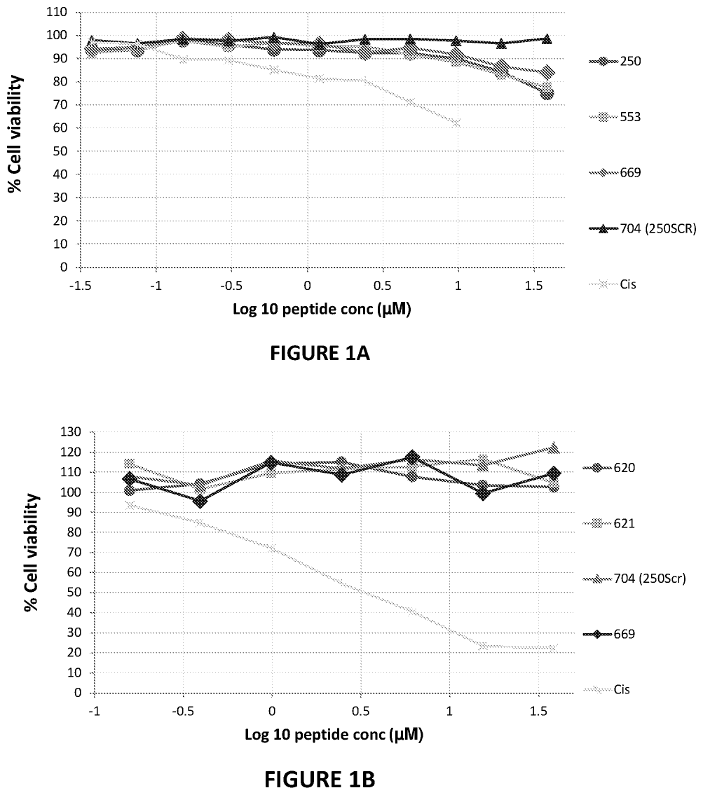

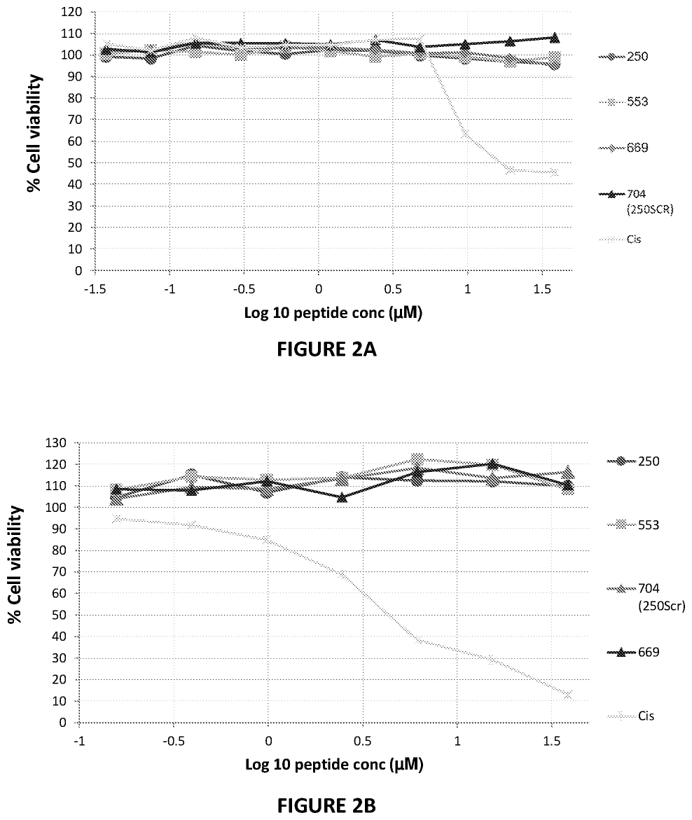

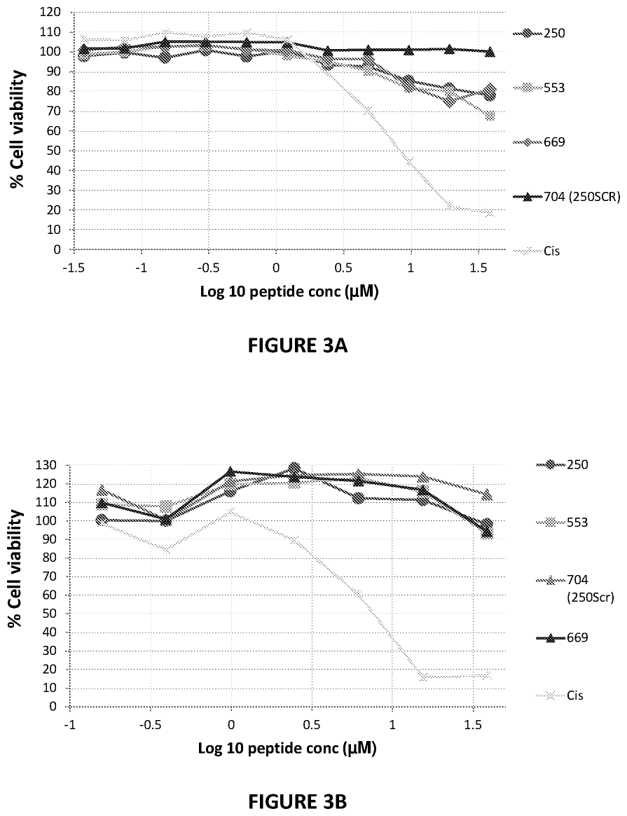

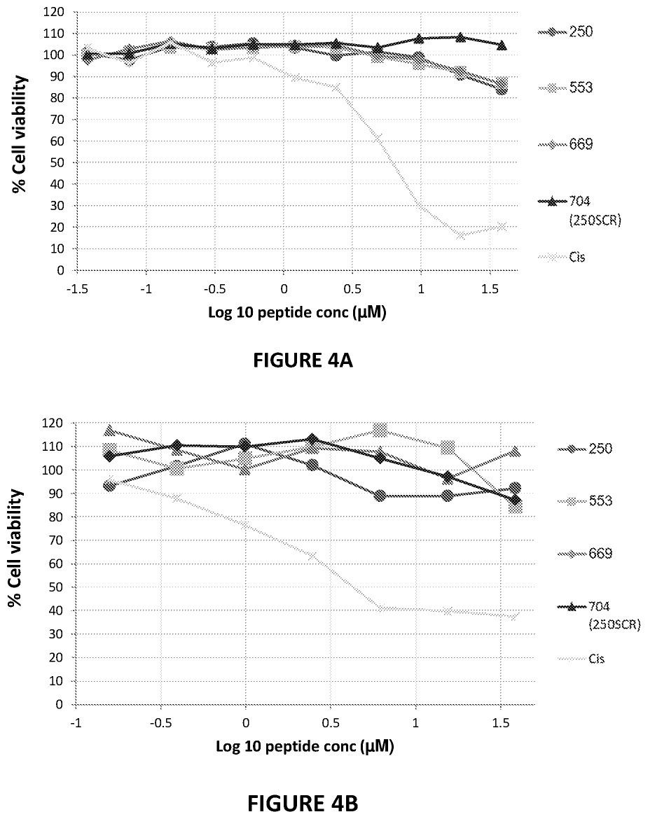

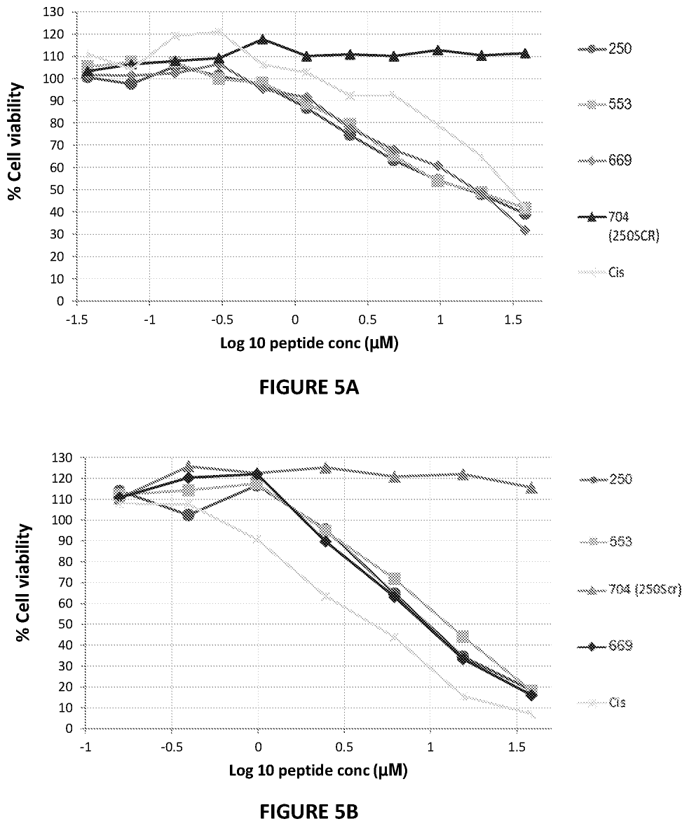

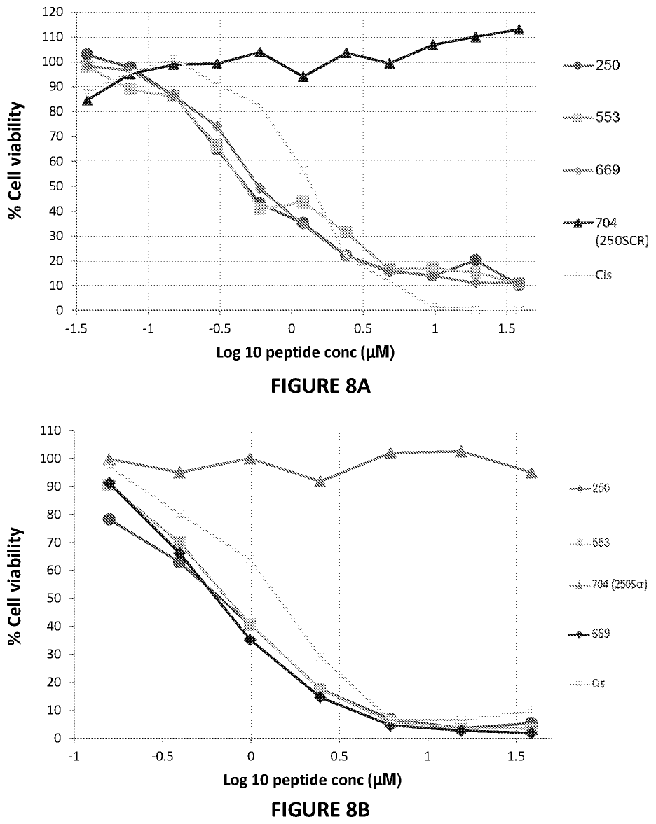

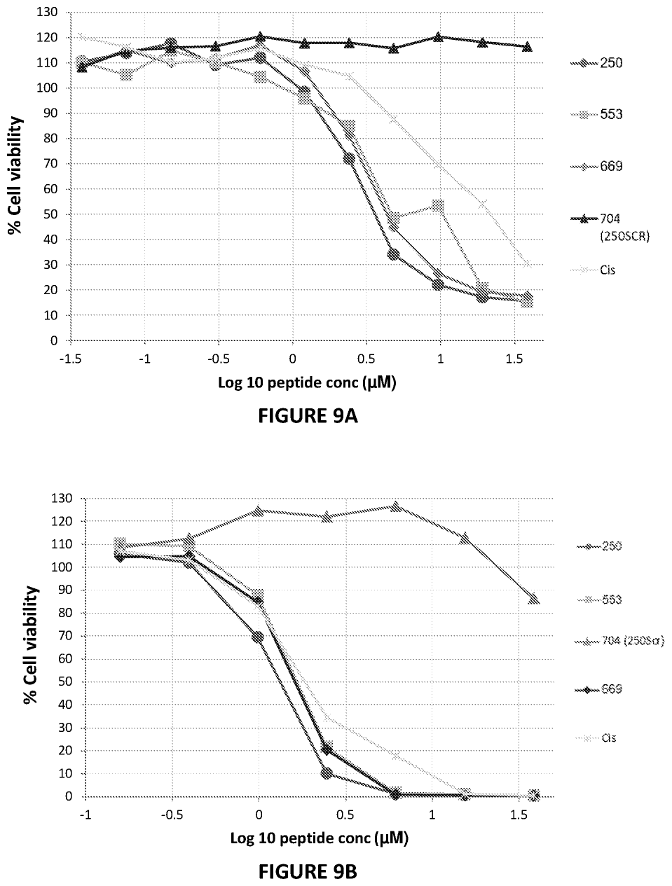

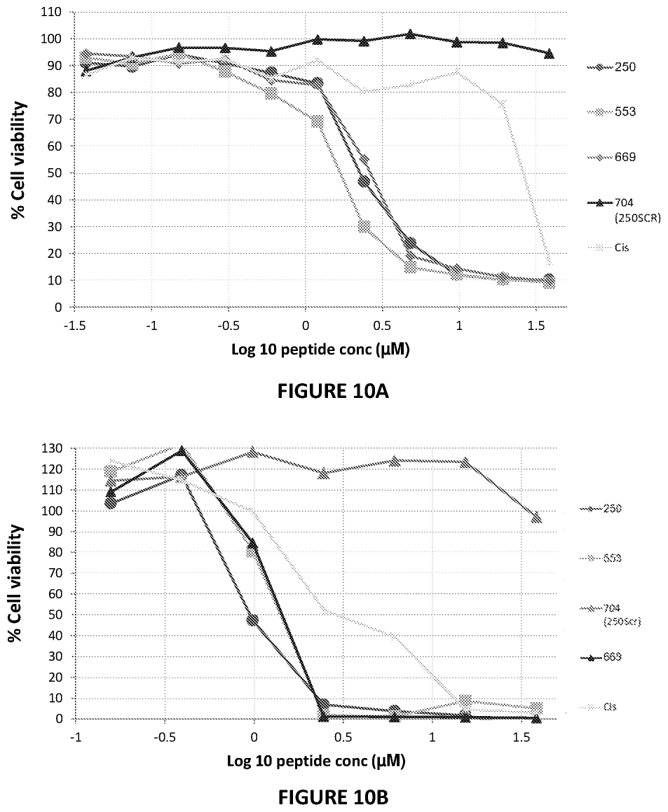

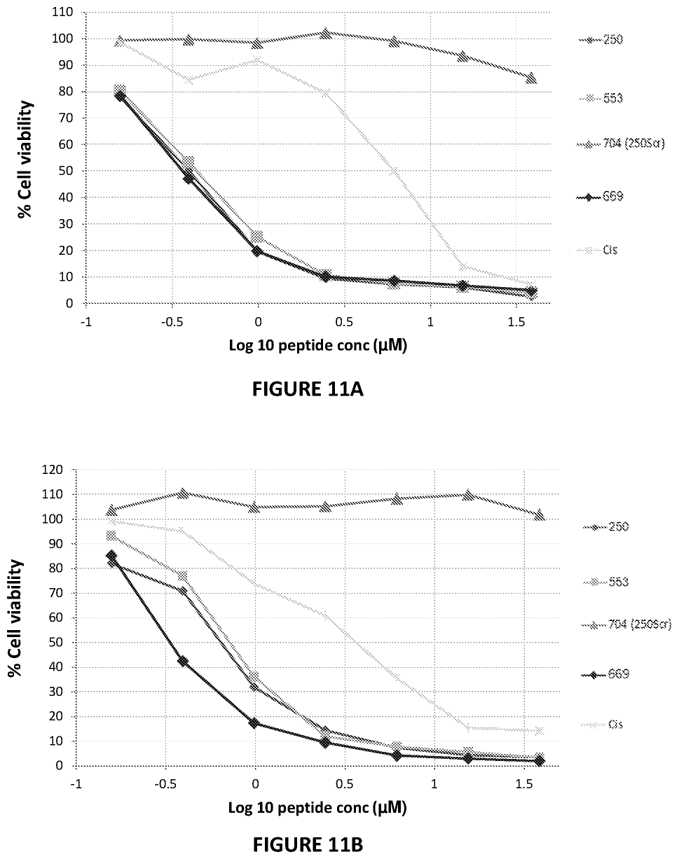

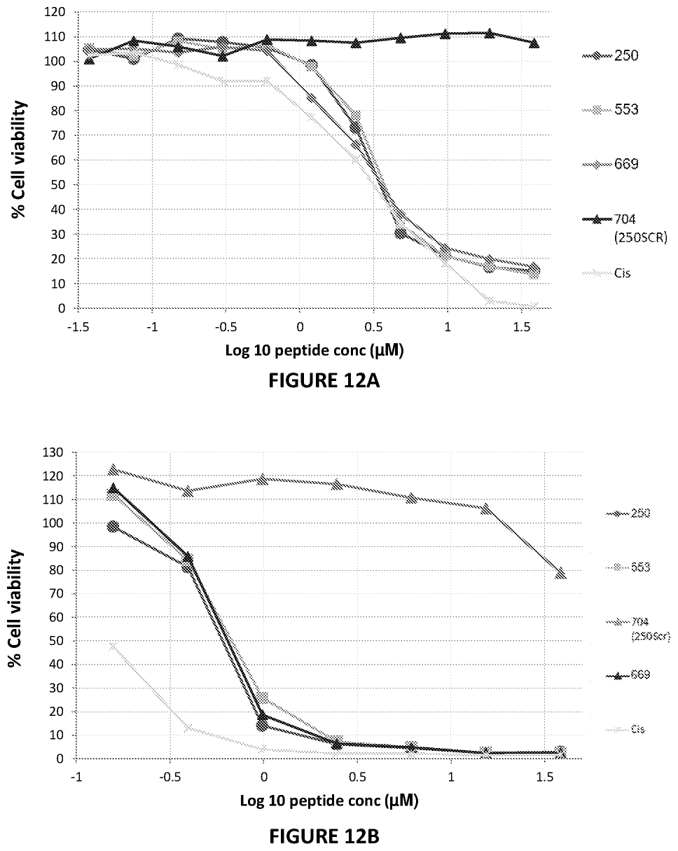

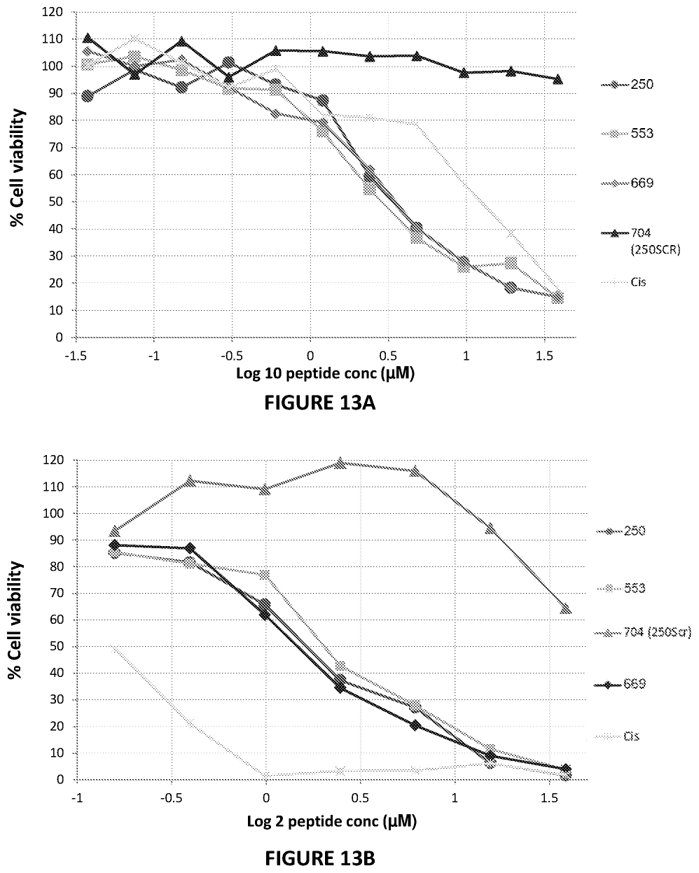

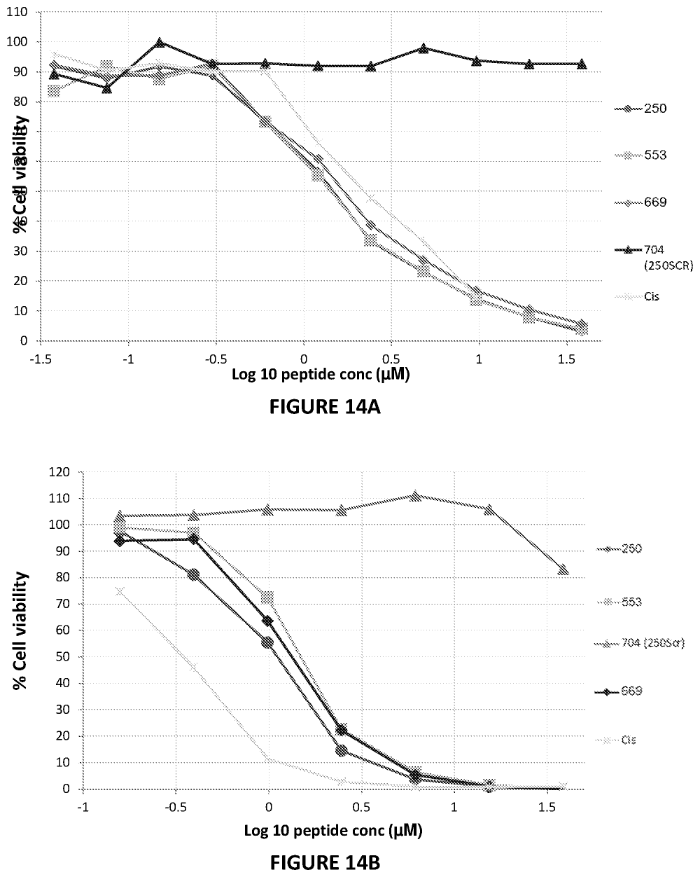

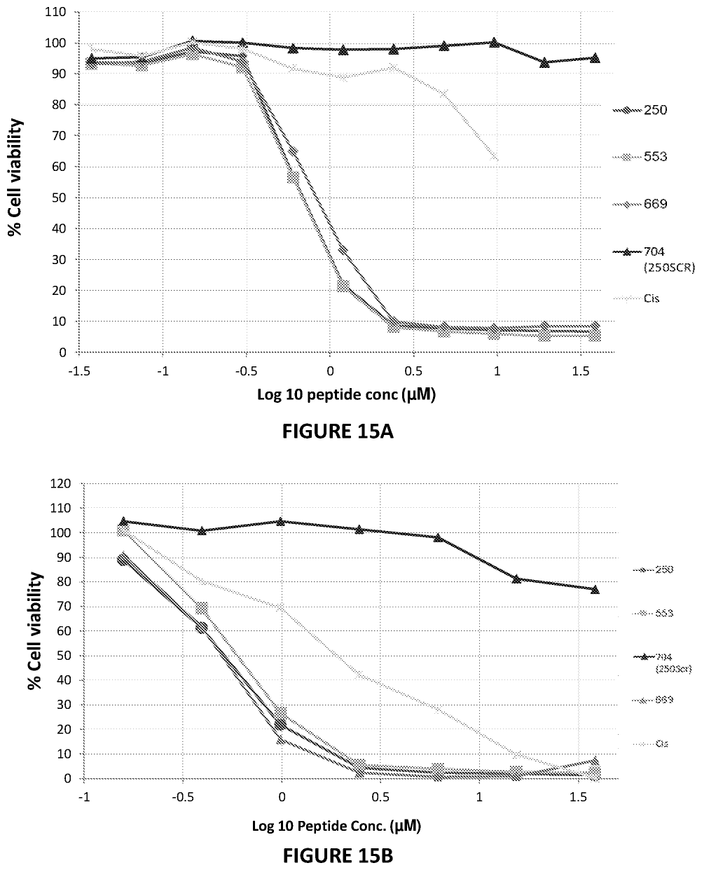

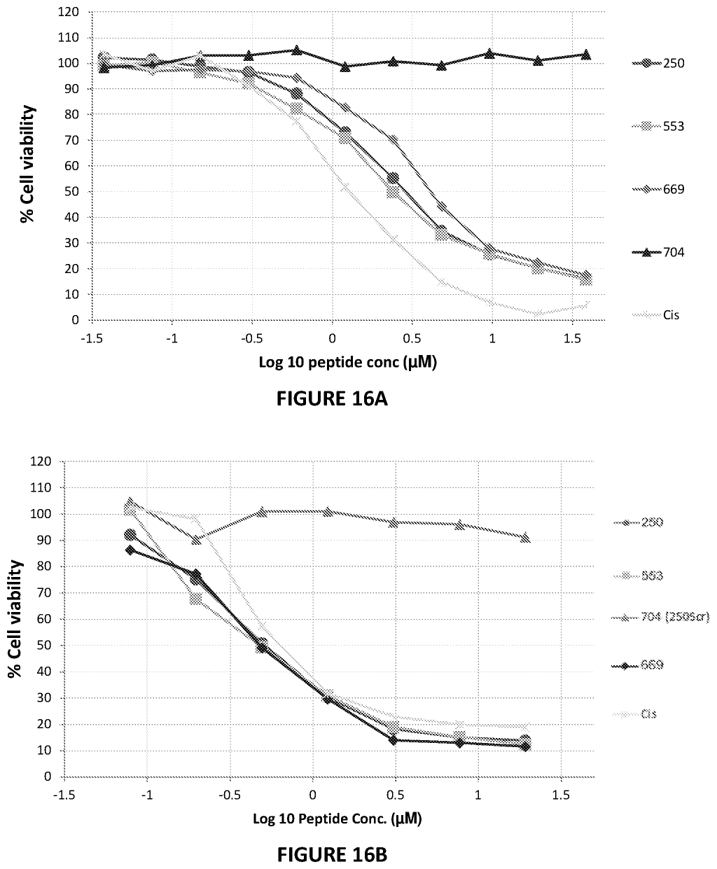

FIGS. 1-16 show the results obtained in a panel of cell lines. The cell lines can be divided into 3 groups: (1) non-transformed cells (FIGS. 1-4), representing normal tissues, (2) cancer cell lines expressing wildtype p53 (wtp53) (FIGS. 5-7), and cancer cell lines expressing mutant p53 (mutp53) (FIGS. 8-16).

As seen in FIGS. 1-4, non-transformed cell lines show no significant reduction in viability in response to peptide treatment at concentrations of up to 40 μM (the highest concentration used). The maximal cell death at a concentration of 40 μM was about 30%. This indicates that non-transformed cells expressing wtp53 and presumably representing normal tissues are quite refractory to peptides of some embodiments of the invention and therefore the overall toxicity of peptide treatment in vivo is expected to be limited.

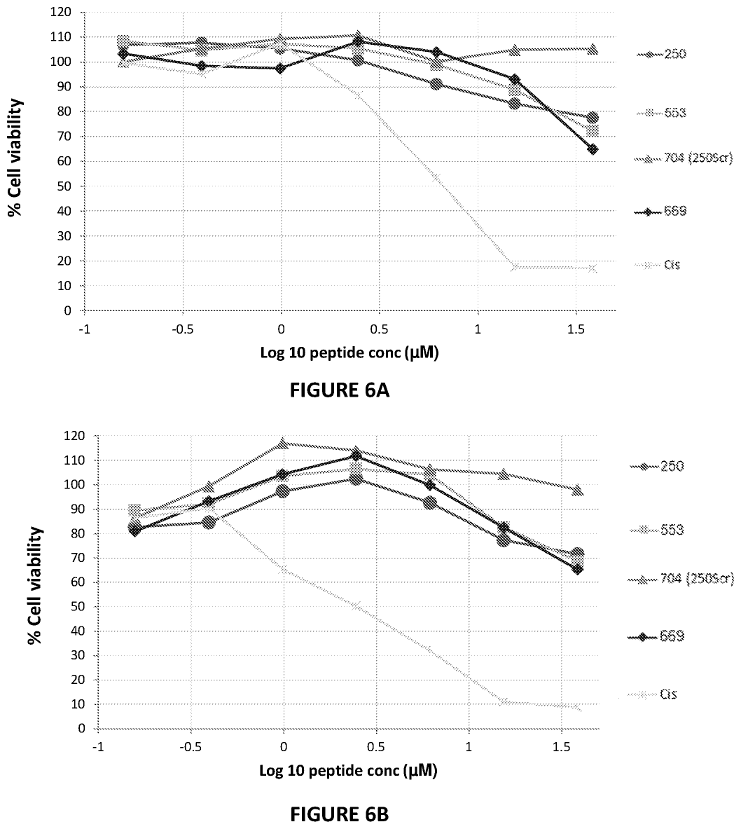

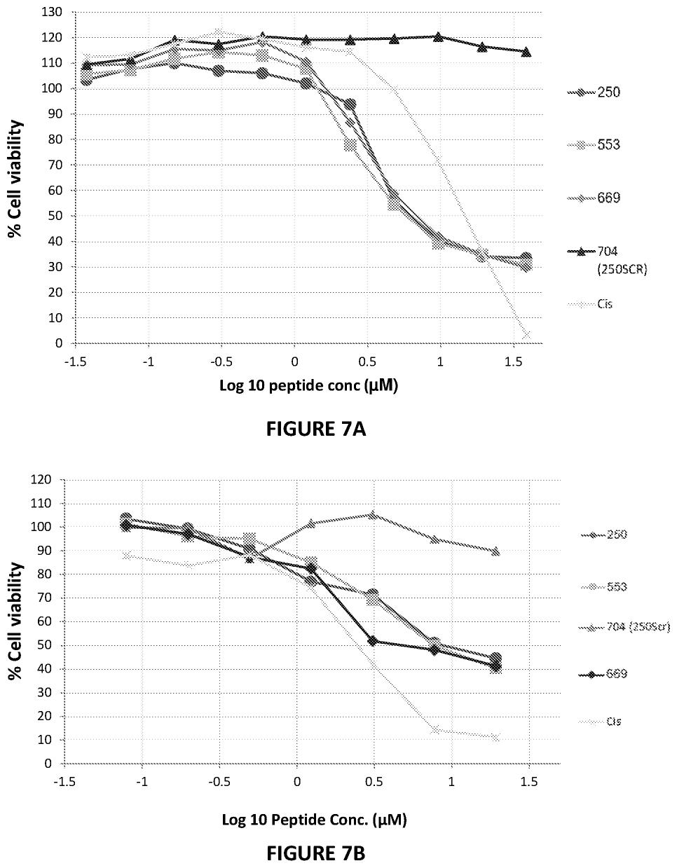

FIGS. 5-7 show peptide treatment response rates of cancer cell lines expressing wild-wtp53. As seen, peptides of some embodiments of the invention exhibit an intermediate effect on the viability of cancer cells expressing wtp53, with IC50 values ranging from 7 μM to over 40 μM. HCT116 human colon carcinoma cells are refractory to peptide treatment (FIG. 6), similar to the non-transformed cells, whereas MCF7 human breast cancer cells and RKO cells show average IC50 values of 14 μM and 7 μM, respectively (FIGS. 5 and 7). The mechanism by which peptides act on wtp53 is not yet clear; however, it is known that in some cancer cells that express wtp53, the protein is dysfunctional despite the absence of mutations. In some cases, this dysfunctionality is due to impaired folding of the protein. It is thus conceivable that peptides of some embodiments of the invention might facilitate the correct folding of dysfunctional wtp53 and restore some of its activity. This opens the possibility for treatment of tumors bearing wtp53.

FIGS. 8-16 show peptide treatment response rates of cancer cell lines expressing mutant p53 (mutp53). As seen, the effect of the peptides varies to some extent between different cell lines; this can be expected, since different cancer cell lines often show very different response rates to a variety of other drugs. Overall, the majority of the tested cell lines exhibit a relatively strong effect of the peptides on the viability of cancer cells expressing mutp53, with IC50 values ranging from 0.204 to 4 μM.

The overall response of cancer cells and in particular the strong response of mutp53-expressing cells relative to lack of significant effect on the viability of non-transformed cells is encouraging, and suggests a relatively large therapeutic window for clinical applications.

### Example 3

### Peptide Design for Improved Efficacy and Stability

A considerable number of pCAP-250 (SEQ ID NO: 24) derivatives were synthesized in an attempt to understand the relationship between sequence, structure and activity of the peptides, and to improve the efficacy of lead peptides. Several lead peptide sequences were identified with improved activity in terms of killing of the tested cancer cell lines. One of those lead peptide sequences is pCAP-553 myr-RRHSvP(L-Dab)PD (see PCT Publication No. WO2017/134671). This peptide was selected for further development since it has better solubility at physiological pH and has also shown improved efficacy relatively to pCAP-250, in viability assays with some of the tested cell lines.

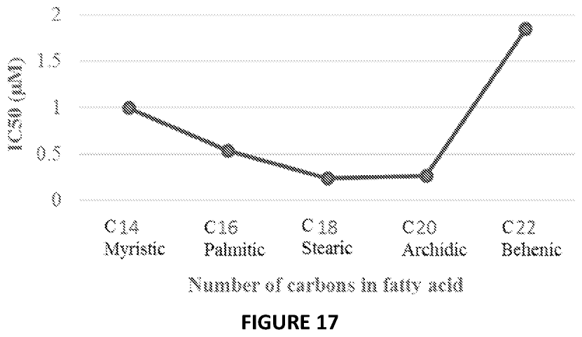

The IC50 tables presented in FIGS. 17-20 are from several representative experiments performed on cell lines expressing several p53 hotspot mutations, as well as control non-transformed cells:

| | | |
|---|---|---|
| RXF-393 | Renal carcinoma | p53R175H |
| SW-480 | Colon carcinoma | p53R273H, P309S |
| PC9 | Lung cancer | p53R248Q |
| MIA PaCa-2 | Pancreatic cancer | p53R248W |
| PANC1 | Pancreatic cancer | p53R273H |
| WI38 | non-transformed fibroblasts | wtp53 |
| MRC5 | non-transformed fibroblasts | wtp53 |

Peptide Modification Strategies

Fatty acid position. Lead peptides were modified with N-terminal myristic acid to enable the penetration of peptides across the cell membrane. Although myristic acid is usually the default choice for cell permeability, additional options were explored of longer fatty acids and to examine the effect on peptide efficacy in terms of cancer cell killing. A standard crystal violet viability test was performed on human renal carcinoma RXF-393 cells, comparing the original pCAP-553, which has myristic acid (C14), with 4 derivative peptides modified with longer fatty acid chains: palmitic (C16), stearic (C18), arachidonic (C20) and behenic (C22).

As seen from FIG. 17, longer fatty acid chains increased the efficacy of the peptide; a peptide modified with palmitic acid (C16, pCAP-673, SEQ ID NO: 3) had a two-fold increase in efficacy relative to pCAP-553, and the peptide modified with stearic acid (C18, pCAP-674, SEQ ID NO: 27) had a four-fold increase in efficacy, with an IC50 of 0.25 μM. This may be due to increased cell penetration. However, further elongation of the fatty acid chain was counterproductive, increasing IC50 values and also exerting higher toxic effects on non-transformed cells (data not shown).

Therefore, further development was done with the stearic acid-modified peptide pCAP-674 (SEQ ID NO: 27).

The use of peptides with N-terminal stearic acid might have an additional important added value: stearic acid conjugates were shown to have two-fold stronger binding to serum albumin compared with myristic acid conjugates, which may increase the half-life of the peptides in vivo.

Proline residue position—Based on alanine scanning and molecular dynamics analyses, it is believed that the proline residue at the 6^{th} position from the N-terminal is critical for the peptide's 3D structure and provides some rigidity required for peptide binding and activity. Therefore, it was hypothesized that a change in orientation or tetrahedral angle might increase the affinity of the peptide for p53. Six new peptides were synthesized replacing this proline residue with proline analogs (pCAPs 708-712, and pCAP-716, SEQ ID NOS: 6-10 and 14, respectively). This set of peptides was tested for activity on a panel of cell lines expressing mutp53, and compared it to pCAP-674 (SEQ ID NO: 27) and to the scrambled sequence peptides pCAP-721 (SEQ ID NO: 19) and pCAP-722 (SEQ ID NO: 20). Most of the proline replacements derivatives did not show an increase in activity. However, pCAP-708 (D-proline) and pCAP-716 (Dimethyl-proline, SEQ ID NO: 14) showed consistently modestly improved activity over pCAP-674 (SEQ ID NO: 27) with IC50 values ranging between 0.12-1.3 μM.

Peptide cyclization—Simulations of peptide folding in solution showed that the N-terminus and C-terminus of the peptide are in close proximity. Therefore, cyclization of the peptide might stabilize this folding and also might provide the peptide with rigidity that increase its ability to exert an effect on protein structure. Peptide cyclization may also have a beneficial effect on the half-life of the peptide within the body.

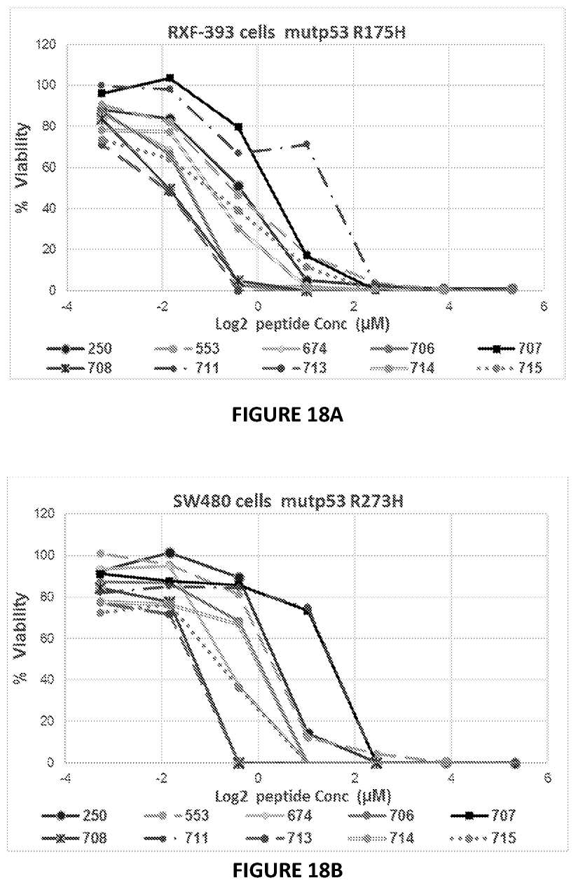

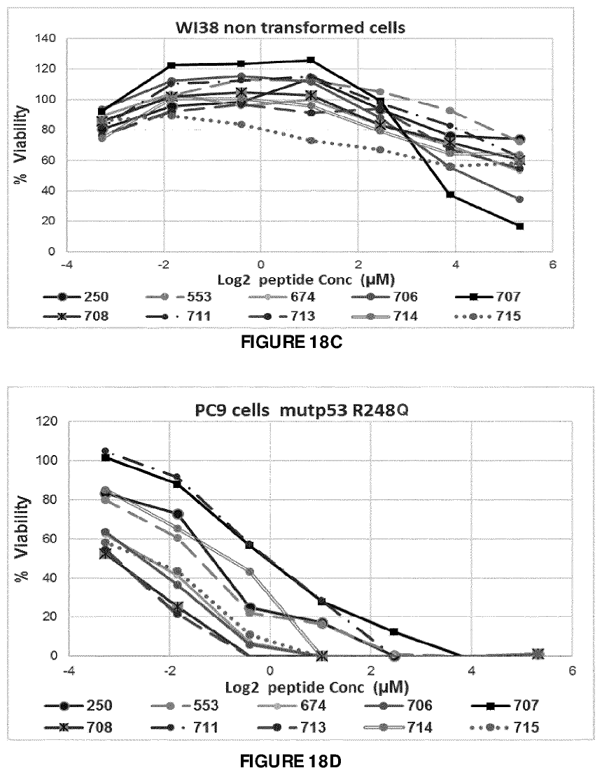

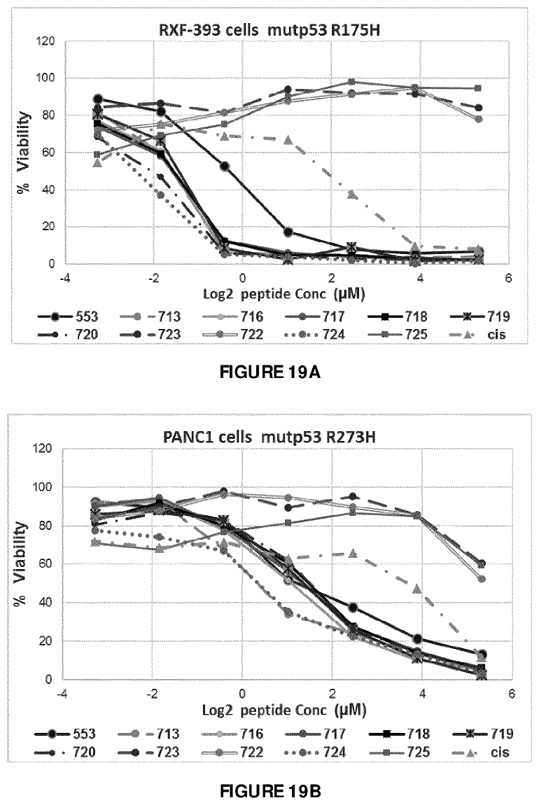

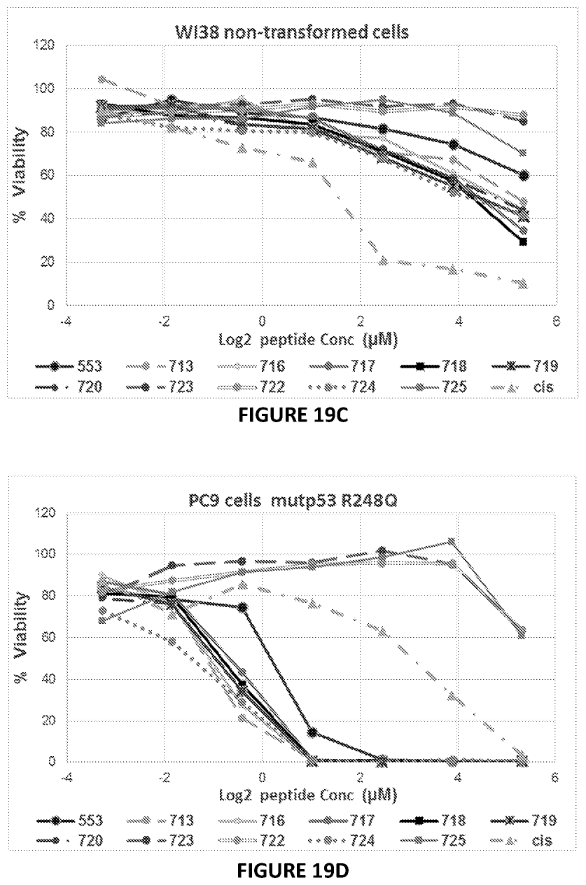

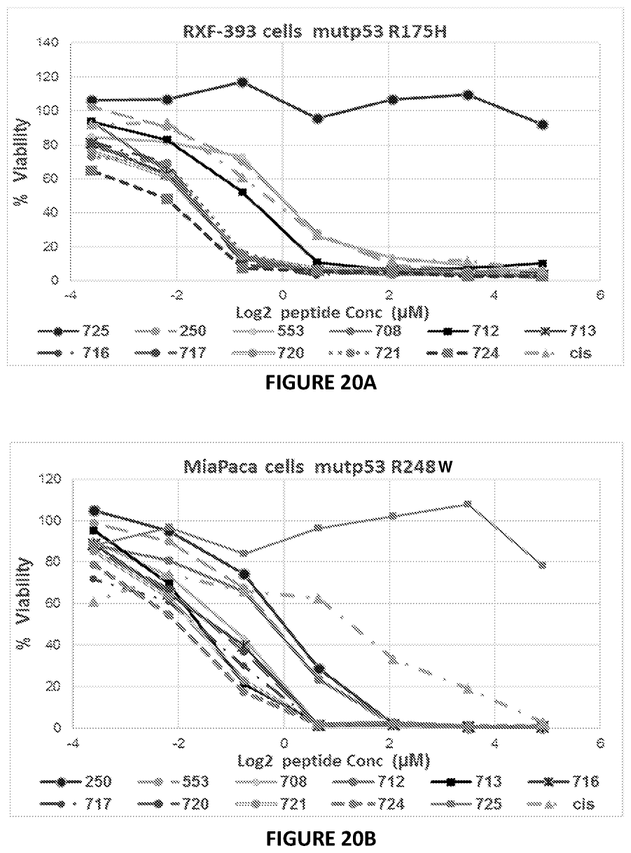

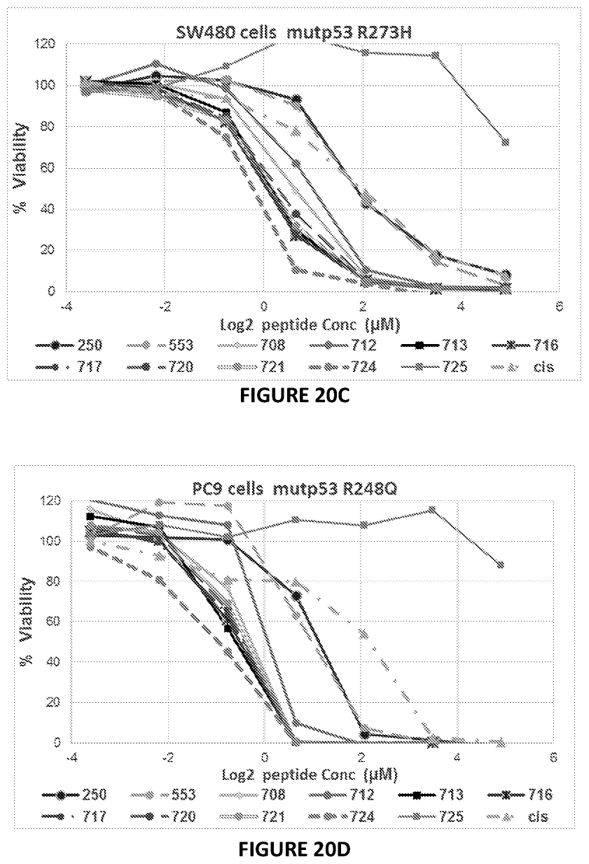

Four new peptides were synthesized with cyclization of a lysine side chain to the C-terminus of the peptide (pCAPs 713-715, SEQ ID NOs: 11-13, respectively), including a scrambled cyclic sequence as a control (pCAP 725, SEQ ID NO: 23). As shown in FIGS. 18-20, of the four cyclic peptides synthesized, pCAP-713 showed the highest activity with a 1.5-3 folds increase over pCAP-674 (non-cyclic peptide) and 4-10 folds over the original pCAP-553. These two peptides are consistently showing sub-micromolar IC50 values of 0.1-1 μM. The IC50 values (in μM) calculated from the graphs presented in FIGS. 18-20 are:

| | FIG. 18 | RXF | SW 480 | PC9 | WI38 |
|---|---|---|---|---|---|
| pCAP | Sequence | IC50 | IC50 | IC50 | IC50 |
| 250 | myr-RRHSTPHPD | 0.89 | 1.2 | 0.51 | >40 |
| 553 | myr-RRHSvP(DAB)PD | 0.81 | 1.12 | 0.42 | >40 |
| 674 | str-RRHSvP(DAB) | 0.4 | 0.6 | 0.21 | 36 |
| | PD-NH₂ | | | | |
| 706 | arc-RRHSvP(Dab) | 0.4 | 0.8 | 0.26 | 15 |
| | PD-NH₂ | | | | |
| 707 | Beh-RRHSvP(Dab) | 1.5 | 3 | 1.5 | 12 |
| | PD-NH₂ | | | | |
| 708 | str-RRHSvp(DAB) | 0.27 | 0.4 | 0.12 | 32 |
| | PD-NH₂ | | | | |
| 711 | str-RRHSv(Oic) | 2.6 | 3 | 1.6 | >40 |
| | (DAB)PD-NH₂ | | | | |
| 713 | str-RRHSKP(DAB) | 0.24 | 0.42 | 0.108 | >40 |
| | PD-Cyc | | | | |
| 714 | str-RRKSvP(DAB) | 0.57 | 1.09 | 0.8 | >40 |
| | PD-Cyc | | | | |
| 715 | str-RRKHSvP(DAB) | 0.48 | 0.54 | 0.25 | >40 |
| | PD Cyc | | | | |

| | FIG. 19 | RXF | PANCI | PC9 | WI38 |
|---|---|---|---|---|---|
| pCAP | Sequence | IC50 | IC50 | IC50 | IC50 |
| 553 | myr-RRHSvP(DAB) | 1.8 | 2.1 | 1.36 | >40 |
| | PD | | | | |
| 713 | str-RRHSKP(DAB) | 0.4 | 1.05 | 0.48 | 30 |
| | PD-Cyc | | | | |
| 716 | str-RRHSv(Dmp) | 0.42 | 2 | 0.63 | 33 |
| | (DAB)PD-NH₂ | | | | |
| 717 | str-RRHSvP(Dab) | 0.38 | 2.15 | 0.78 | 35 |
| | PD-NH₂ | | | | |
| 718 | str-RRHSvP(Dab) | 0.37 | 2.21 | 0.72 | >40 |
| | PD-NH₂ ion ex | | | | |
| 719 | str-RRHSk(tic) | 0.46 | 2.18 | 0.57 | 29 |
| | (Dab)PD-NH₂ | | | | |
| 720 | str-RRHSvp(Dab) | 0.24 | 1.24 | 0.59 | 32 |
| | PDGEA | | | | |
| 722 | str-DPHvSPR(Dab) | >40 | >40 | >40 | >40 |
| | R-NH₂ | | | | |
| 723 | str-RDPHvSPR | >40 | >40 | >40 | >40 |
| | (Dab-NH₂ | | | | |
| 724 | str-RRHSkp(Dab) | 0.16 | 1.02 | 0.42 | 32 |
| | PD-Cyc | | | | |
| 725 | str-DPHKSPR(Dab) | >40 | >40 | >40 | >40 |
| | R-Cyc | | | | |

| | FIG. 20 | RXF 393 | MIAPaca | PC9 | SW-480 |
|---|---|---|---|---|---|
| pCAP | Sequence | IC50 | IC50 | IC50 | IC50 |
| 250 | myr-RRHSTPHPD | 1 | 1 | 1 | 5 |
| 553 | myr-RRHSvP | 1 | 1.2 | 1 | 5 |
| | (DAB)PD | | | | |
| 708 | str-RRHSvp(DAB) | 0.4 | 0.5 | 0.5 | 2 |
| | PD-NH₂ | | | | |
| 712 | str-RRHSv(Tic) | 0.6 | 1 | 1.2 | 1.5 |
| | (DAB)PD-NH₂ | | | | |
| 713 | str-RRHSKP(DAB) | 0.3 | 0.3 | 0.6 | 0.9 |
| | PD-Cyc | | | | |
| 716 | str-RRHSv(Dmp) | 0.3 | 0.3 | 0.7 | 1 |
| | (DAB)PD-NH₂ | | | | |
| 717 | str-RRHSvP | 0.3 | 0.3 | 0.7 | 1.2 |
| | (Dab)PD-NH₂ | | | | |
| 720 | str-RRHSvp | 0.3 | 0.3 | 0.7 | 1.2 |
| | (Dab)PDGEA | | | | |
| 721 | str-RRHSvp | 0.3 | 0.3 | 0.8 | 1.2 |
| | (Dab)PdGR | | | | |
| 724 | str-RRHSkp | 0.2 | 0.25 | 0.5 | 0.65 |
| | (Dab)PD-Cyc | | | | |
| 725 | str-DPHKSPR | >30 | >30 | >30 | >30 |
| | (Dab)R-Cyc | | | | |

Targeting of peptides to tumors and tumor vasculature—The RGDX (SEQ ID NO: 47, wherein X is absent or is any amino acid residue) and DGEA (SEQ ID NO: 54) motifs are known to interact with integrins overexpressed on the tumor neovasculature and tumor cells and therefore are widely used to target drugs to tumor sites. The incorporation of these motifs into peptide analog sequences was therefore examined, for future clinical applications. Active tumor targeting can significantly increase the concentrations of peptides in tumor sites (up to 20-fold) and therefore enhance their efficacy in vivo. Two peptides were tested with targeting motifs conjugated to the C-terminus of the lead peptide: pCAP-720 (SEQ ID NO: 18) with a DGEA motif and pCAP-721 (SEQ ID NO: 19), with dGR motif (retro inverso strategy for RGD motif). Both peptides showed comparable activity to pCAP-717 (SEQ ID NO: 15) and pCAP-708 (SEQ ID NO: 6), indicating that the addition of a targeting sequence at the C-terminus did not reduce their activity.

Combination of modifications leading to enhanced activity—The present results indicate that pCAPs 708 (SEQ ID NO: 6), 712 (SEQ ID NO: 10), 716 (SEQ ID NO: 14) and 713 (SEQ ID NO: 11) have the highest activity. The modifications of pCAP-708 (SEQ ID NO: 6) and pCAP-713 (SEQ ID NO: 11) were combined, synthesizing a cyclic peptide with D-proline (pCAP-724, SEQ ID NO: 22). pCAP-724 showed a modest increase in activity compared with pCAP-713 (SEQ ID NO: 11) and pCAP-708 (SEQ ID NO: 6). Hence, there is seemingly an additive contribution of D-proline to the efficacy of the cyclic peptide.

### CONCLUSIONS

From the analysis of the results of the above experiments, the following could be concluded:

Stearic fatty acid is preferable to myristic acid, lowering the IC50 for most cell lines by 2-3 folds. Therefore, it was decided to continue with stearic acid peptides (pCAP-674, SEQ ID NO: 27, or pCAP-717, SEQ ID NO: 15).

Proline position and derivatization—most of the replacements for proline did not show an increase in activity. However, peptide pCAP-708 (D-proline, SEQ ID NO: 6) showed consistently improved activity over pCAP-674 (SEQ ID NO: 27), with IC50 ranging 0.12-1.3 μM.

Tumor targeting—both peptides containing tumor targeting motifs (pCAP-720 and pCAP-721, SEQ ID Nos 18 and 19 respectively) show comparable activity to pCAP-717 (SEQ ID NO: 15) and 708 (SEQ ID NO: 6), indicating that the addition of targeting sequence in the C-terminus did not reduce their activity.

Peptide cyclization—out of the four cyclic peptides synthesized, pCAP-713 (SEQ ID NO: 11) and pCAP-724 (SEQ ID NO: 22) show the highest activity with a 1.5-3 folds increase over pCAP-717 (SEQ ID NO: 15) and a 4-10 folds over the original pCAP-553 (SEQ ID NO: 2), with IC50 values of 0.1-1 μM.

### Example 4

### Pharmacokinetic Profile of Peptide Analogs

The pharmacokinetic profile of a drug is one of the most important factors since it determines the drug's absorption, distribution, metabolism, and excretion from the body, which together with pharmacodynamics influence the drug's dosing, benefit and adverse effects,

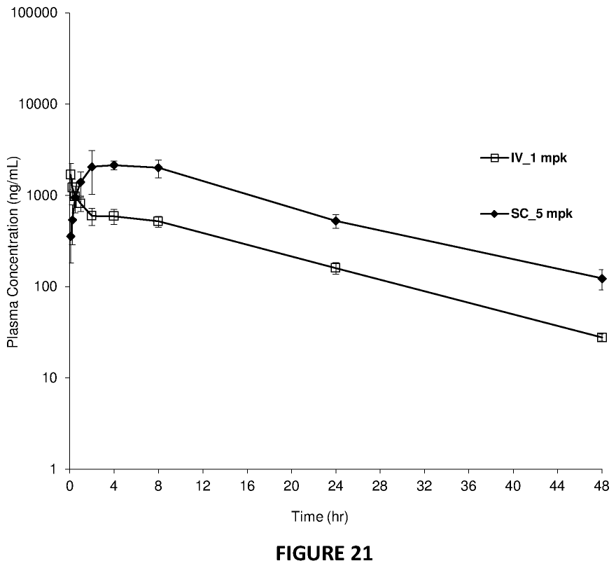

FIG. 21 shows a pharmacokinetic analysis of pCAP-724 containing an N-terminal stearic acid, performed by LC-MS analysis of peptide concentration in the plasma. Each point represents the average from 3 mice. When administered subcutaneously into mice, pCAP-724 levels reach a maximal concentration after 2 h and a show a half-life (T1/2) of about 10 h. This is significantly higher than the T1/2 measured previously for the N-terminally myristoylated pCAP-250 (SEQ ID NO: 24), and might be due to the stronger binding of stearic fatty acid to albumin. From the PK study, it is estimated that a continuous long-acting administration of 20 mg/kg/day of pCAP-724 (SEQ ID NO: 22) will yield a steady state concentration in the blood of about 7-10 μM.

### Example 5

### Preclinical Evaluation of Lead Peptide Analogs

To investigate the efficacy of the lead peptides in vivo, a representative cancer cell line-based xenograft model was employed. The selection of the in vivo model was based on the peptide/cell line combination that yielded the strongest effects in culture: human renal carcinoma RXF-393 cells. The peptide analog pCAP-724 (SEQ ID NO: 22), administered by subcutaneous injections and sustained subcutaneous release, was compared to the control scrambled peptide pCAP-722 (SEQ ID NO: 20). Tumor growth was monitored by luciferase activity, measured in an IVIS200® Spectrum system that combines 2D optical and 3D optical tomography in one platform. The system uses leading optical technology for preclinical imaging research and development, ideal for non-invasive longitudinal monitoring of disease progression in living animals

The In Vivo Protocol

The RXF-393 renal cell carcinoma (RCC) cell line, endogenously expressing p53R175H, and stably expressing Luciferase was utilized.
1. 0.75×10⁶ cells in 50% HBSS 50% Cultrex (1000) were injected subcutaneously, into both hips of athymic Nude-Foxn1nu mice (6 weeks old).
2. Tumor growth over time was measured by live imaging, using the IVIS2000 system. Exposure time was calibrated to 20 sec and peak luminescence values were taken for each tumor.
3. 21 and 24 days after injection of cells, when tumors reached visible size (50-100 mm³) and an average IVIS signal of 10⁷ (100-fold over threshold), the mice were randomly divided into several groups (5 or 6 mice per each group), according to the mode of administration and peptides:
   Subcutaneous injections, performed 2 days a week for 3-4 weeks (20 mg/Kg). Two injections will be given for each day of treatment 16 h interval to allow effective level of peptide for 24 h. One group was treated with pCAP-724 (SEQ ID NO: 22) and one group was treated with the control, scrambled peptide pCAP-722 (SEQ ID NO: 20). Treatment was initiated at day 21 post-cancer cells injection.
   Subcutaneous continuous administration with Alzet mini-pumps (20 mg/Kg/Day). 14 days duration pumps were used. One group was treated with pCAP-724 (SEQ ID NO: 22). A bolus injection of 20 mg/Kg peptide was given at the time of pump implantation, to enable fast build-up of an effective concentration. Treatment was initiated at day 24.

| Mice/group | Treatment | Peptide |
|---|---|---|
| 6 | SC injection twice a week | pCAP-722 (SEQ ID NO: 20) |
| 5 | SC injection twice a week | pCAP-724 (SEQ ID NO: 22) |
| 6 | SC pump - continuous | pCAP-724 (SEQ ID NO: 22) |

4. Tumor growth over time was measured by live imaging, twice a week. The experiment was conducted until tumors reached the maximal allowed size of 1 cm³, at which time mice were sacrificed, tumors extracted, measured, photographed, and weighed.

Results

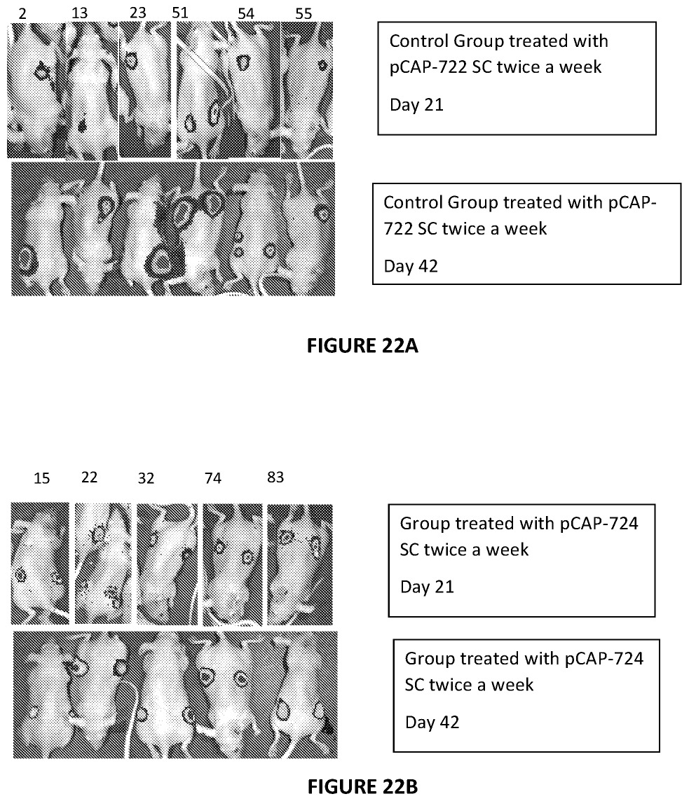

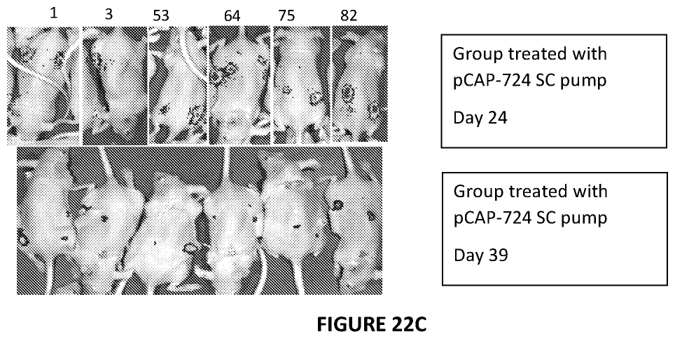

As seen in FIG. 22, at day 21, when tumors reached visible size (50-100 mm³) and an average IVIS signal of 10⁷ (100-fold over threshold), mice were randomly divided into 3 groups for subcutaneous injection. The rest of the mice were subjected at day 24 to continuous subcutaneous administration using an Alzet minipumps. As seen from the figure, the IVIS signal at the beginning of the treatments was comparable between the different groups. However, at the end of the experiment (day 39 and day 42) clear differences were evident between the groups. In particular, the mice treated with Alzet minipumps displayed considerably smaller tumors, as determined by both IVIS (FIG. 22) and by tumor weight (FIG. 24).

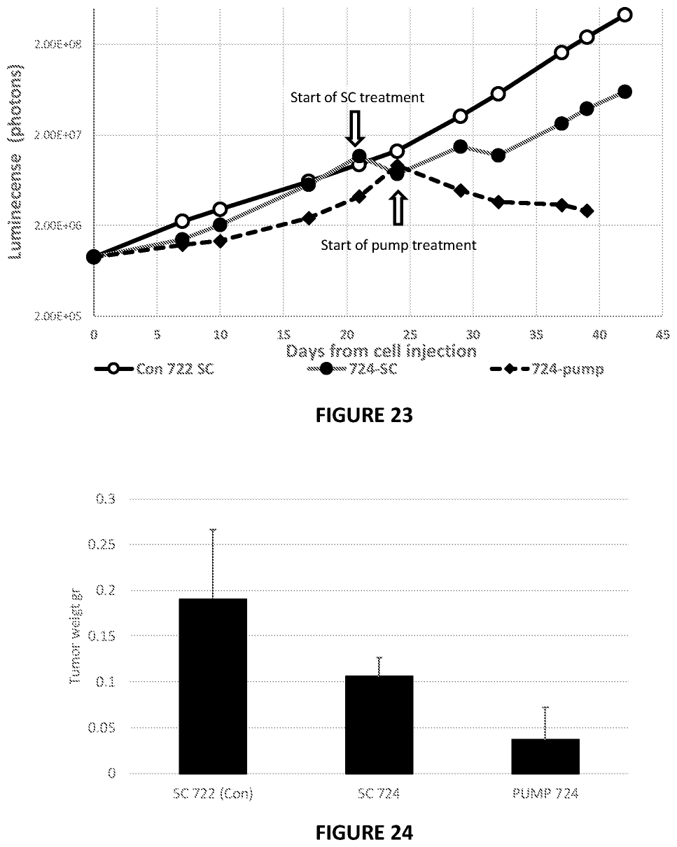

FIG. 23 is a quantitative illustration of the average IVIS signal of each of the treatment groups over time. As seen from the figure, the tumors grew exponentially until the beginning of the treatment. While the control peptide injected group continued its exponential growth, the pCAP-724 subcutaneous injections slowed down tumor growth for the first 10 days. The two groups treated with pCAP-724 using Alzet mini pumps showed a remarkable effect on tumor growth, with the pCAP-724 group showing a 5-fold decrease in tumor signal within 14 days.

These results underscore the importance of long-acting continuous administration of the peptides and peptide analogs for maximum efficacy.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-32. (canceled)

33. A peptide analog comprising the amino acid sequence Z₁-RRHSX₁X₂(Dab)PD-Z₂ (SEQ ID NO: 72) wherein:
X₁ is selected from D-Valine (v), L-Lysine (K) and D-Lysine (k);
X₂ is selected from the group consisting of D-Proline (p), diaminobutyric acid (Dab), Di-methyl proline (Dmp), 4-tetrahydroisoquinoline-3-carboxylic acid (Tic), (S)-(−)-Indoline-2-carboxylic acid (Idc), Pipecolic acid (Pip), and octahydroindolecarboxylic acid (Oic);
Z₁ is a fatty acid residue comprising 16 to 19 carbon atoms;
Z₂ denotes the carboxy terminus of the peptide analog which is: an unmodified C-terminus; an amidated C-terminus, connected to a side chain of an amino acid residue to form a cyclic peptide; or
connected to a targeting moiety, and
wherein the peptide at least partially reactivates a mutant p53 protein.

34. The peptide analog of claim 33, wherein X₁X₂ is selected from the group consisting of: vp, kp, v(Dmp), v(Idc), v(Pip), v(Oic), and v(Tic).

35. The peptide analog of claim 33, wherein X₁ is selected from K and k and the peptide analog is cyclized by connecting the carboxy terminus with the free amine group of the side chain of the K or k residues.

36. The peptide analog of claim 33, wherein the peptide analog is selected from the group consisting of:
pCAP724-str-RRHSkp(Dab)PD
(SEQ ID NO: 22, Cyclized by connecting
the D-Lys side chain to the carboxy
terminus);
PCAP708-
str-RRHSvp(Dab)PD-NH₂;
(SEQ ID NO: 6)
pCAP720-
str-RRHSvp(Dab)PDGEA;
(SEQ ID NO: 18)
pCAP721-
str-RRHSvp(Dab)PdGR;
(SEQ ID NO: 19)
pCAP716
str-RRHSv(Dmp)(Dab)PD-NH₂;
(SEQ ID NO: 14)
pCAP709-
str-RRHSv(Idc)(Dab)PD-NH₂;
(SEQ ID NO: 7)
pCAP710-
str-RRHSv(Pip)(Dab)PD-NH₂;
(SEQ ID NO: 8)
pCAP711
str-RRHSv(Oic)(Dab)PD-NH₂;
(SEQ ID NO: 9)
and
pCAP712-
str-RRHSv(Tic)(Dab)PD-NH₂.
(SEQ ID NO: 10)

37. The peptide analog of claim 33, wherein X₁ is v, X₂ is selected from Dmp, Idc, Pip, Oic, and Tic and the peptide analog is selected from the group consisting of:
pCAP716-
(SEQ ID NO: 14)
str-RRHSv(Dmp)(Dab)PD-NH₂;
pCAP709-
(SEQ ID NO: 7)
str-RRHSv(Idc)(Dab)PD-NH₂;
pCAP710-
(SEQ ID NO: 8)
str-RRHSv(Pip)(Dab)PD-NH₂;
pCAP711-
(SEQ ID NO: 9)
str-RRHSv(Oic)(Dab)PD-NH₂;
and
pCAP712-
(SEQ ID NO: 10)
str-RRHSv(Tic)(Dab)PD-NH₂.

38. The peptide analog of claim 33, wherein the peptide analog is the cyclic peptide pCAP-724 having the amino acid sequence str-RRHSkp(Dab)PD (SEQ ID NO: 22, Cyclized by connecting the D-Lys side chain to the carboxy terminus).

39. The peptide analog of claim 33, wherein the peptide analog is up to 15 amino acids long.

40. The peptide analog of claim 33, wherein the peptide analog consists of 9 to 12 amino acid residues and a fatty acid residue comprising 16 to 19 carbon atoms.

41. The peptide analog of claim 33, comprising at least one modification selected from a cyclization, C-terminal amidation, and conjugation of a targeting moiety.

42. The peptide analog of claim 41, wherein the modification is cyclization, and the cyclization is between the carboxy terminus and an amino acid side chain.

43. The peptide analog of claim 33, comprising a targeting moiety connected to the carboxy terminus.

44. The peptide analog of claim 43, wherein said targeting moiety is selected from: PDGED (SEQ ID NO: 70) or a retro-inverso orientation thereof; DGEA (SEQ ID NO: 54) or a retro-inverso orientation thereof; and RGDX (SEQ ID NO: 47) or a retro-inverso orientation thereof, wherein X is absent or is any amino acid residue.

45. The peptide analog of claim 33, wherein the peptide analog at least partially changes the conformation of said mutant p53 protein to a conformation of a wild-type (WT) p53 protein.

46. The peptide analog of claim 33, wherein the peptide analog at least partially restores an activity of said mutant p53 protein to the activity of a WT p53 protein.

47. The peptide analog of claim 46, wherein said activity is at least one of:
reducing viability of cells expressing said mutant p53 protein;
promoting apoptosis of cells expressing said mutant p53 protein; and
binding to a p53 consensus DNA binding element in cells expressing said mutant p53 protein.

48. A pharmaceutical composition comprising a therapeutically effective amount of at least one peptide analog according to claim 33, and a pharmaceutically active excipient, diluent, or carrier.

49. A method of treating cancer, comprising administering to a subject in need thereof a pharmaceutical composition according to claim 48, thereby treating the cancer.

50. The method of claim 49, wherein the cancer is selected from the group consisting of: breast cancer, colon cancer, ovarian cancer, and lung cancer.

51. The method of claim 49, wherein the cancer is a metastatic cancer.

52. The method of claim 49, wherein the pharmaceutical composition is administered systemically by injection or infusion.


SEQUENCE LISTING (73 sequences)

SEQ ID NO: 1 - protein, 9 aa, Artificial sequence
  Synthetetic peptide
  misc_feature (1)..(1): may be conjugated to a hydrophobic moiety selected from the group consisting of palmitoyl (C16); phtanoyl ((CH3)4); heptadecanoyl (C17); stearoyl (C18) and nonadecanoyl (C19)
  misc_feature (1)..(9): may be cyclized ( between side chain to the C terminus)
  misc_feature (1)..(9): may have a targeting moiety
  misc_feature (5)..(5): X  can be any amino acid
  misc_feature (6)..(6): X is selected from the group consisting of D-Proline, Dab, L-Proline, Dmp, DMP and  tic
  misc_feature (7)..(7): X is   Diaminobutyric Acid (DAB)
RRHSXXXPD

SEQ ID NO: 2 - protein, 9 aa, Artificial sequence
  Synthetetic peptide
  MISC_FEATURE (1)..(1): MYRISTATE group attached
  MISC_FEATURE (5)..(5): D- Valine
  MISC_FEATURE (7)..(7): X is L- Diaminobutyric Acid (L-DAB)
RRHSVPXPD

SEQ ID NO: 3 - protein, 9 aa, Artificial sequence
  Synthetetic peptide
  MISC_FEATURE (1)..(1): Palmitic acid  conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHSVPXPD

SEQ ID NO: 4 - protein, 9 aa, Artificial sequence
  Synthetetic peptide
  MISC_FEATURE (1)..(1): Arachidic acid conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHSVPXPD

SEQ ID NO: 5 - protein, 9 aa, Artificial sequence
  Synthetetic peptide
  MISC_FEATURE (1)..(1): Behenic acid conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHSVPXPD

SEQ ID NO: 6 - protein, 9 aa, Artificial sequence
  Synthetetic peptide
  MISC_FEATURE (1)..(1): Stearic acid conjugated
  MISC_FEATURE (5)..(5): D- Valine
  MISC_FEATURE (6)..(6): D-proline
  MISC_FEATURE (7)..(7): X is  Diaminobutyric Acid (DAB)
  MISC_FEATURE (9)..(9): C-terminal amidation
RRHSVPXPD

SEQ ID NO: 7 - protein, 9 aa, Artificial sequence
  Synthetetic peptide
  MISC_FEATURE (1)..(1): Stearic acid conjugated
  MISC_FEATURE (5)..(5): D- Valine
  misc_feature (6)..(6): x is (S)-(-)-Indoline-2-carboxylic acid
  MISC_FEATURE (7)..(7): X is  Diaminobutyric Acid (DAB)
  MISC_FEATURE (9)..(9): C-terminal amidation
RRHSVXXPD

SEQ ID NO: 8 - protein, 9 aa, Artificial sequence
  Synthetetic peptide
  MISC_FEATURE (1)..(1): Stearic acid conjugated
  MISC_FEATURE (5)..(5): D- Valine
  misc_feature (6)..(6): x is Pipecolic acid
  MISC_FEATURE (7)..(7): X is  Diaminobutyric Acid (DAB)
  MISC_FEATURE (9)..(9): C-terminal amidation
RRHSVXXPD

SEQ ID NO: 9 - protein, 9 aa, Artificial sequence
  Synthetetic peptide
  MISC_FEATURE (1)..(1): Stearic acid conjugated
  MISC_FEATURE (5)..(5): D- Valine
  misc_feature (6)..(6): x is octahydroindolecarboxylic acid(Oic)
  MISC_FEATURE (7)..(7): X is  Diaminobutyric Acid (DAB)
RRHSVXXPD

SEQ ID NO: 10 - protein, 9 aa, Artificial sequence
  Synthetetic peptide
  MISC_FEATURE (1)..(1): Stearic acid conjugated
  MISC_FEATURE (5)..(5): D- Valine
  misc_feature (6)..(6): x is 4-tetrahydroisoquinoline-3-carboxylic acid (Tic)
  MISC_FEATURE (7)..(7): X is  Diaminobutyric Acid (DAB)
  MISC_FEATURE (9)..(9): C-terminal amidation
RRHSVXXPD

SEQ ID NO: 11 - protein, 9 aa, Artificial sequence
  Synthetetic peptide
  MISC_FEATURE (1)..(1): Stearic acid conjugated
  misc_feature (1)..(9): Cyclization of Lysine side chain to C terminal carboxyl
  MISC_FEATURE (7)..(7): X is  Diaminobutyric Acid (DAB)
RRHSKPXPD

SEQ ID NO: 12 - protein, 9 aa, Artificial sequence
  Synthetetic peptide
  MISC_FEATURE (1)..(1): Stearic acid conjugated
  misc_feature (1)..(9): Cyclization of Lysine side chain to C terminal carboxyl
  MISC_FEATURE (5)..(5): D- Valine
  MISC_FEATURE (7)..(7): X is  Diaminobutyric Acid (DAB)
RRKSVPXPD

SEQ ID NO: 13 - protein, 10 aa, Artificial sequence
  Synthetetic peptide
  MISC_FEATURE (1)..(1): Stearic acid conjugated
  misc_feature (1)..(10): Cyclization of Lysine side chain to C terminal carboxyl
  MISC_FEATURE (6)..(6): D- Valine
  MISC_FEATURE (8)..(8): X is  Diaminobutyric Acid (DAB)
RRKHSVPXPD

SEQ ID NO: 14 - protein, 9 aa, Artificial sequence
  Synthetetic peptide
  MISC_FEATURE (1)..(1): Stearic acid conjugated
  MISC_FEATURE (5)..(5): D- Valine
  misc_feature (6)..(6): x is  Di-methyl proline (Dmp)
  MISC_FEATURE (7)..(7): X is  Diaminobutyric Acid (DAB)
  MISC_FEATURE (9)..(9): C-terminal amidation
RRHSVXXPD

SEQ ID NO: 15 - protein, 9 aa, Artificial sequence
  Synthetetic peptide
  MISC_FEATURE (1)..(1): Stearic acid conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHSVPXPD

SEQ ID NO: 16 - skipped

SEQ ID NO: 17 - protein, 9 aa, Artificial sequence
  Synthetetic peptide
  MISC_FEATURE (1)..(1): Stearic acid conjugated
  MISC_FEATURE (5)..(5): D- lysine
  misc_feature (6)..(6): x is 4-tetrahydroisoquinoline-3-carboxylic acid (Tic)
  MISC_FEATURE (7)..(7): X is  Diaminobutyric Acid (DAB)
  MISC_FEATURE (9)..(9): C-terminal amidation
RRHSKXXPD

SEQ ID NO: 18 - protein, 12 aa, Artificial sequence
  Synthetetic peptide
  MISC_FEATURE (1)..(1): Stearic acid conjugated
  MISC_FEATURE (5)..(5): D- Valine
  MISC_FEATURE (6)..(6): D- Proline
  MISC_FEATURE (7)..(7): X is  Diaminobutyric Acid (DAB)
RRHSVPXPDGEA

SEQ ID NO: 19 - protein, 11 aa, Artificial sequence
  Synthetetic peptide
  MISC_FEATURE (1)..(1): Stearic acid conjugated
  MISC_FEATURE (5)..(5): D- Valine
  MISC_FEATURE (6)..(6): D- Proline
  MISC_FEATURE (7)..(7): X is  Diaminobutyric Acid (DAB)
RRHSVPXPDGR

SEQ ID NO: 20 - protein, 9 aa, Artificial sequence
  Synthetetic peptide
  MISC_FEATURE (1)..(1): Stearic acid conjugated
  MISC_FEATURE (4)..(4): D- Valine
  MISC_FEATURE (8)..(8): X is  Diaminobutyric Acid (DAB)
  MISC_FEATURE (9)..(9): C-terminal amidation
DPHVSPRXR

SEQ ID NO: 21 - protein, 9 aa, Artificial sequence
  Synthetetic peptide
  MISC_FEATURE (1)..(1): Stearic acid conjugated
  MISC_FEATURE (5)..(5): D- Valine
  MISC_FEATURE (9)..(9): X is  Diaminobutyric Acid (DAB)
  MISC_FEATURE (9)..(9): C-terminal amidation
RDPHVSPRX

SEQ ID NO: 22 - protein, 9 aa, Artificial sequence
  Synthetetic peptide
  MISC_FEATURE (1)..(1): Stearic acid conjugated
  MISC_FEATURE (1)..(9): Cyclization of Lysine side chain to C terminal carboxyl
  MISC_FEATURE (5)..(5): D- Lysine
  MISC_FEATURE (6)..(6): D- Proline
  MISC_FEATURE (7)..(7): X is  Diaminobutyric Acid (DAB)
RRHSKPXPD

SEQ ID NO: 23 - protein, 9 aa, Artificial sequence
  Synthetetic peptide
  MISC_FEATURE (1)..(1): Stearic acid conjugated
  MISC_FEATURE (1)..(9): Cyclization of Lysine side chain to C terminal carboxyl
  MISC_FEATURE (8)..(8): X is  Diaminobutyric Acid (DAB)
DPHKSPRXR

SEQ ID NO: 24 - protein, 9 aa, Artificial sequence
  Synthetetic peptide
RRHSTPHPD

SEQ ID NO: 25 - protein, 9 aa, Artificial sequence
  Synthetetic peptide
  MISC_FEATURE (1)..(1): MYRISTATE group conjugated
  MISC_FEATURE (6)..(6): X is  alpha-aminoisobutyric acid
  MISC_FEATURE (9)..(9): C-terminal amidation
RRHSTXHAD

SEQ ID NO: 26 - protein, 9 aa, Artificial sequence
  Synthetetic peptide
  MISC_FEATURE (1)..(1): MYRISTATE group conjugated
DPHTSPRHR

SEQ ID NO: 27 - protein, 9 aa, Artificial sequence
  Synthetetic peptide
  MISC_FEATURE (1)..(1): Stearic acid conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHSVPXPD

SEQ ID NO: 28 - protein, 9 aa, Artificial sequence
  Synthetetic peptide
  MISC_FEATURE (1)..(1): Stearic acid conjugated
  MISC_FEATURE (1)..(9): Cyclization of Dab side chain to C terminal carboxyl
  MISC_FEATURE (5)..(5): X is  Diaminobutyric Acid (DAB)
  MISC_FEATURE (7)..(7): X is  Diaminobutyric Acid (DAB)
RRHSXPXPD

SEQ ID NO: 29 - protein, 10 aa, Artificial sequence
  Synthetetic peptide
  MISC_FEATURE (1)..(1): MYRISTATE group conjugated
  MISC_FEATURE (7)..(7): X is  Diaminobutyric Acid (DAB)
RRHSEPXPDH

SEQ ID NO: 30 - protein, 10 aa, Artificial sequence
  Synthetetic peptide
  MISC_FEATURE (1)..(1): MYRISTATE group conjugated
  MISC_FEATURE (7)..(7): X is  Diaminobutyric Acid (DAB)
RRHSEPXPAH

SEQ ID NO: 31 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (6)..(6): X is  alpha-aminoisobutyric acid
  MISC_FEATURE (7)..(7): X is  Diaminobutyric Acid (DAB)
RRHSVXXPD

SEQ ID NO: 32 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHSVPXPD

SEQ ID NO: 33 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RAHSVPXPD

SEQ ID NO: 34 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRASVPXPD

SEQ ID NO: 35 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHAVPXPD

SEQ ID NO: 36 - protein, 9 aa, Artificial sequence
  Synthetetic peptide
  MISC_FEATURE (1)..(1): MYRISTATE group conjugated
  MISC_FEATURE (7)..(7): X is  Diaminobutyric Acid (DAB)
  MISC_FEATURE (9)..(9): C-terminal amidation
RRHSAPXPD

SEQ ID NO: 37 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHSVAXPD

SEQ ID NO: 38 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (9)..(9): C-terminal amidation
RRHSVPAPD

SEQ ID NO: 39 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHSVPXAD

SEQ ID NO: 40 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (7)..(7): X is  Diaminobutyric Acid (DAB)
  MISC_FEATURE (9)..(9): C-terminal amidation
RRHSVPXPA

SEQ ID NO: 41 - protein, 9 aa, Artificial sequence
  Synthetetic peptide
  MISC_FEATURE (1)..(1): MYRISTATE group conjugated
  MISC_FEATURE (5)..(5): D- Threonine
  MISC_FEATURE (7)..(7): X is  Diaminobutyric Acid (DAB)
  MISC_FEATURE (9)..(9): C-terminal amidation
RRHSTPXPD

SEQ ID NO: 42 - protein, 4 aa, Artificial sequence
  Synthetetic peptide
DGEA

SEQ ID NO: 43 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (7)..(7): X is  ornithine (Orn)
  MISC_FEATURE (9)..(9): C-terminal amidation
RRHSVPXPD

SEQ ID NO: 44 - protein, 9 aa, Artificial sequence
  Synthetetic peptide
  MISC_FEATURE (1)..(1): MYRISTATE group conjugated
  MISC_FEATURE (5)..(5): D- Valine
  MISC_FEATURE (9)..(9): C-terminal amidation
RRHSVPKPD

SEQ ID NO: 45 - protein, 9 aa, Artificial sequence
  Synthetetic peptide
  MISC_FEATURE (1)..(1): MYRISTATE group conjugated
  MISC_FEATURE (5)..(5): D- Serine
  MISC_FEATURE (7)..(7): X is  Diaminobutyric Acid (DAB)
  MISC_FEATURE (9)..(9): C-terminal amidation
RRHSSPXPD

SEQ ID NO: 46 - protein, 9 aa, Artificial sequence
  Synthetetic peptide
  MISC_FEATURE (1)..(1): MYRISTATE group conjugated
  MISC_FEATURE (5)..(5): FITC labeled
  MISC_FEATURE (7)..(7): X is  Diaminobutyric Acid (DAB)
  MISC_FEATURE (9)..(9): C-terminal amidation
RRHSKPXPD

SEQ ID NO: 47 - protein, 4 aa, Artificial sequence
  Synthetetic peptide
  MISC_FEATURE (4)..(4): X is absent or selected from any aino acid residue
RGDX

SEQ ID NO: 48 - protein, 5 aa, Artificial sequence
  Synthetetic peptide
GRGDS

SEQ ID NO: 49 - protein, 4 aa, Artificial sequence
  Synthetetic peptide
RGDS

SEQ ID NO: 50 - protein, 6 aa, Artificial sequence
  Synthetetic peptide
KQAGDV

SEQ ID NO: 51 - protein, 5 aa, Artificial sequence
  Synthetetic peptide
YIGSR

SEQ ID NO: 52 - protein, 4 aa, Artificial sequence
  Synthetetic peptide
REDV

SEQ ID NO: 53 - protein, 15 aa, Artificial sequence
  Synthetetic peptide
GTPGPQGIAGQRGVV

SEQ ID NO: 54 - protein, 5 aa, Artificial sequence
  Synthetetic peptide
PDGEA

SEQ ID NO: 55 - protein, 5 aa, Artificial sequence
  Synthetetic peptide
IKVAV

SEQ ID NO: 56 - protein, 10 aa, Artificial sequence
  Synthetetic peptide
RNIAEIIKDI

SEQ ID NO: 57 - protein, 10 aa, Artificial sequence
  Synthetetic peptide
KHIFSDDSSE

SEQ ID NO: 58 - protein, 5 aa, Artificial sequence
  Synthetetic peptide
VPGIG

SEQ ID NO: 59 - protein, 7 aa, Artificial sequence
  Synthetetic peptide
FHRRIKA

SEQ ID NO: 60 - protein, 4 aa, Artificial sequence
  Synthetetic peptide
KRSR

SEQ ID NO: 61 - protein, 13 aa, Artificial sequence
  Synthetetic peptide
KFAKLAARLYRKA

SEQ ID NO: 62 - protein, 14 aa, Artificial sequence
  Synthetetic peptide
KHKGRDVILKKDVR

SEQ ID NO: 63 - protein, 8 aa, Artificial sequence
  Synthetetic peptide
YKKIIKKL

SEQ ID NO: 64 - protein, 19 aa, Artificial sequence
  Synthetetic peptide
NSPVNSKIPKACCVPTELS

SEQ ID NO: 65 - protein, 6 aa, Artificial sequence
  Synthetetic peptide
AIAPGL

SEQ ID NO: 66 - protein, 4 aa, Artificial sequence
  Synthetetic peptide
  MISC_FEATURE (4)..(4): X is absent or is selected form any amino acid residue
VRNX

SEQ ID NO: 67 - protein, 9 aa, Artificial sequence
  Synthetetic peptide
AAAAAAAAA

SEQ ID NO: 68 - protein, 7 aa, Artificial sequence
  Synthetetic peptide
  MISC_FEATURE (1)..(1): Acetyl group conjugated
GCRDGPQ

SEQ ID NO: 69 - protein, 9 aa, Artificial sequence
  Synthetetic peptide
GIWGQDRCG

SEQ ID NO: 70 - protein, 5 aa, Artificial sequence
  Synthetetic peptide
PDGED

SEQ ID NO: 71 - protein, 10 aa, Artificial Sequence
  Synthetic peptide
  LIPID (1)..(1): a fatty acid residue comprising 16 to 19 carbon atoms attached to the N-terminus
  MISC_FEATURE (5)..(5): selected from D-Valine (v), L-Lysine (K) and D-Lysine (k)
  MISC_FEATURE (6)..(6): selected from L-Proline (P), D-Proline (p), di amino butyric acid (Dab), Di-methyl proline (Dmp), 4-tetrahydroisoquinoline-3-carboxylic acid (Tic), (S)-(-)-Indoline-2-carboxylic acid (Idc), Pipecolic acid (Pip),
  MOD_RES (7)..(7): Dbu
  MOD_RES (9)..(9): an unmodified C-terminus; an amidated C-terminus, connected to a side chain of an amino acid residue to form a cyclic peptide; or connected to a targeting moiety
  misc_feature (10)..(10): Xaa can be any naturally occurring amino acid
RRHSXXXPDX

SEQ ID NO: 72 - protein, 10 aa, Artificial Sequence
  Synthetic peptide
  LIPID (1)..(1): a fatty acid residue comprising 16 to 19 carbon atoms
  MISC_FEATURE (5)..(5): selected from D-Valine (v), L-Lysine (K) and D-Lysine (k)
  MISC_FEATURE (6)..(6): selected from: D-Proline (p), di amino butyric acid (Dab), Di-methyl proline (Dmp), 4-tetrahydroisoquinoline-3- carboxylic acid (Tic), (S)-(-)-Indoline-2-carboxylic acid (Idc), Pipecolic acid (Pip), and octahydroindolecarboxylic acid (Oic)
  MOD_RES (7)..(7): Dbu
  MISC_FEATURE (9)..(9): an unmodified C-terminus; an amidated C-terminus, connected to a side chain of an amino acid residue to form a cyclic peptide; or connected to a targeting moiety
  misc_feature (10)..(10): Xaa can be any naturally occurring amino acid
RRHSXXXPDX

SEQ ID NO: 73 - protein, 9 aa, Artificial Sequence
  Synthetic peptide
  LIPID (1)..(1): a fatty acid residue comprising at least 16 carbon atoms
  MISC_FEATURE (5)..(5): D-Lysine (k) or L-Lysine (K)
  MISC_FEATURE (5)..(9): Cyclization between the side chain of the Lysine or D-Lysine residue and the carboxy terminus
  MISC_FEATURE (6)..(6): D-Proline (p) or L-Proline (P)
  MOD_RES (7)..(7): Dbu
RRHSXXXPD